SEQUENCE LISTING (126 sequences)

SEQ ID NO: 1 - DNA, 22 bp, Artificial Sequence
  primer BacEN-F
gccaccatgggatgttctttag

SEQ ID NO: 2 - DNA, 21 bp, Artificial Sequence
  primer BacEN-B
gtactcagaaacaactttggc

SEQ ID NO: 3 - DNA, 30 bp, Artificial Sequence
  Primer LF1
cttgaccttgccacagaggactattagagg

SEQ ID NO: 4 - DNA, 30 bp, Artificial Sequence
  Primer LR1
cagtgacacttaccgcattgacaagcacgc

SEQ ID NO: 5 - DNA, 30 bp, Artificial Sequence
  Primer RF1
cctcgatatacagaccgataaaacacatgc

SEQ ID NO: 6 - DNA, 30 bp, Artificial Sequence
  Primer RR1
agtcagtcagaaacaactttggcacatatc

SEQ ID NO: 7 - DNA, 20 bp, Artificial Sequence
  primer EL1
ccatatacgcatcgggttga

SEQ ID NO: 8 - DNA, 25 bp, Artificial Sequence
  primer ER1
ttaaagtttaggtcgagtaaagcgc

SEQ ID NO: 9 - DNA, 14 bp, Human
ttaaagaaacacag

SEQ ID NO: 10 - DNA, 14 bp, Human
ttaataaaggggtt

SEQ ID NO: 11 - DNA, 14 bp, Human
ttaaagctccaaaa

SEQ ID NO: 12 - DNA, 14 bp, Human
ttaaaaaaatttat

SEQ ID NO: 13 - DNA, 14 bp, Human
ttaaagaatcatgg

SEQ ID NO: 14 - DNA, 14 bp, Human
ttaatacaacttgc

SEQ ID NO: 15 - DNA, 14 bp, Human
ttaaaacggaagtt

SEQ ID NO: 16 - DNA, 14 bp, Human
ttaagtaataataa

SEQ ID NO: 17 - DNA, 14 bp, Human
ttaaaagctaagcc

SEQ ID NO: 18 - DNA, 14 bp, Human
ttaattaatctggg

SEQ ID NO: 19 - DNA, 14 bp, Human
ttaagcctataccc

SEQ ID NO: 20 - DNA, 14 bp, Human
ttaaagtaagaaat

SEQ ID NO: 21 - DNA, 14 bp, Human
ttaaaggaatacca

SEQ ID NO: 22 - DNA, 14 bp, Human
ttaaccctcctaac

SEQ ID NO: 23 - DNA, 14 bp, Human
ttaaagatcaaagt

SEQ ID NO: 24 - DNA, 14 bp, Human
ttaataatttgtcc

SEQ ID NO: 25 - DNA, 14 bp, Human
ttaaagaatggtta

SEQ ID NO: 26 - DNA, 14 bp, Human
ttaaaagaccttta

SEQ ID NO: 27 - DNA, 14 bp, Mouse
ttaagttgtaccaa

SEQ ID NO: 28 - DNA, 14 bp, Mouse
ttaaaggagagact

SEQ ID NO: 29 - DNA, 14 bp, Mouse
ttaactgcccagtg

SEQ ID NO: 30 - DNA, 14 bp, Mouse
ttaacaaaacaaaa

SEQ ID NO: 31 - DNA, 14 bp, Mouse
ttaatcaacaaata

SEQ ID NO: 32 - DNA, 14 bp, Mouse
ttaaagagtcccct

SEQ ID NO: 33 - DNA, 14 bp, Mouse
ttaacaacagataa

SEQ ID NO: 34 - DNA, 14 bp, Mouse
ttaagcaaggtcac

SEQ ID NO: 35 - DNA, 14 bp, Mouse
ttaaaggcatggac

SEQ ID NO: 36 - DNA, 14 bp, Mouse
ttagtgatgcctac

SEQ ID NO: 37 - DNA, 14 bp, Mouse
ttaaggagaaaaag

SEQ ID NO: 38 - DNA, 14 bp, Mouse
ttaaataaaatgtc

SEQ ID NO: 39 - DNA, 14 bp, Mouse
ttaagcttttcgtt

SEQ ID NO: 40 - DNA, 14 bp, Mouse
ttaaagggaccttg

SEQ ID NO: 41 - DNA, 14 bp, Mouse
ttaaacatgttctg

SEQ ID NO: 42 - DNA, 14 bp, Mouse
ttaatcccagcact

SEQ ID NO: 43 - DNA, 14 bp, Mouse
ttaacattccagac

SEQ ID NO: 44 - DNA, 14 bp, Mouse
ttaaaactagctgt

SEQ ID NO: 45 - DNA, 14 bp, Mouse
ttaaaattctggga

SEQ ID NO: 46 - DNA, 14 bp, Mouse
ttaagtgggaaagt

SEQ ID NO: 47 - DNA, 14 bp, Mouse
ttaaatatatgaag

SEQ ID NO: 48 - DNA, 14 bp, Mouse
ttaaagaaataaac

SEQ ID NO: 49 - DNA, 14 bp, Mouse
ttaaaaaataattc

SEQ ID NO: 50 - DNA, 14 bp, Mouse
ttaagaacacaggt

SEQ ID NO: 51 - DNA, 14 bp, Mouse
ttaacaaatgtttg

SEQ ID NO: 52 - DNA, 14 bp, Mouse
ttaaaggaaataag

SEQ ID NO: 53 - DNA, 14 bp, Mouse
ttaacaagagctga

SEQ ID NO: 54 - DNA, 14 bp, Mouse
ttaacagaggcagc

SEQ ID NO: 55 - DNA, 14 bp, Mouse
ttaagatgtgtgtg

SEQ ID NO: 56 - DNA, 14 bp, Mouse
ttaaaatcctacaa

SEQ ID NO: 57 - DNA, 14 bp, Mouse
ttaagcttaactgc

SEQ ID NO: 58 - DNA, 14 bp, Mouse
ttaaattgccttcc

SEQ ID NO: 59 - DNA, 14 bp, Mouse
ttaaagaacaacat

SEQ ID NO: 60 - DNA, 14 bp, Mouse
ttaagaatacatac

SEQ ID NO: 61 - DNA, 14 bp, Mouse
ttaatatctgctat

SEQ ID NO: 62 - DNA, 14 bp, Mouse
ttaaggaggaaagg

SEQ ID NO: 63 - DNA, 14 bp, Mouse
ttaagaggaaatcg

SEQ ID NO: 64 - DNA, 14 bp, Mouse
ttaaaatatcttag

SEQ ID NO: 65 - DNA, 14 bp, Mouse
ttaaataaatttaa

SEQ ID NO: 66 - DNA, 14 bp, Mouse
ttaaatagtagaaa

SEQ ID NO: 67 - DNA, 14 bp, Mouse
ttaaggctaagaat

SEQ ID NO: 68 - DNA, 14 bp, Mouse
ttaaaagcagcatt

SEQ ID NO: 69 - DNA, 14 bp, Mouse
ttaaaaattaattg

SEQ ID NO: 70 - DNA, 14 bp, Mouse
ttaaagtcatgtaa

SEQ ID NO: 71 - DNA, 14 bp, Mouse
gttaaagcatttaa

SEQ ID NO: 72 - DNA, 14 bp, Mouse
ttaaggagaaagat

SEQ ID NO: 73 - DNA, 14 bp, Mouse
ttaaagaacaacaa

SEQ ID NO: 74 - DNA, 14 bp, Mouse
ttaaatagttaaaa

SEQ ID NO: 75 - DNA, 14 bp, Mouse
ttaaataagagttg

SEQ ID NO: 76 - DNA, 14 bp, Mouse
ttaatgagtatgca

SEQ ID NO: 77 - DNA, 14 bp, Mouse
ttaaacccttcgcc

SEQ ID NO: 78 - DNA, 14 bp, Mouse
ttaaggaggaaata

SEQ ID NO: 79 - DNA, 14 bp, Mouse
ttaatgttgaagca

SEQ ID NO: 80 - DNA, 14 bp, Mouse
ttaaccgcacttca

SEQ ID NO: 81 - DNA, 14 bp, Mouse
ttaagatttgtaaa

SEQ ID NO: 82 - DNA, 14 bp, Mouse
ttaagggagaaaag

SEQ ID NO: 83 - DNA, 14 bp, Mouse
ttaagcaggaagca

SEQ ID NO: 84 - DNA, 14 bp, Mouse
ttaataactgtttt

SEQ ID NO: 85 - DNA, 14 bp, Mouse
ttaacgaagtccaa

SEQ ID NO: 86 - DNA, 14 bp, Mouse
ttaaggctagactg

SEQ ID NO: 87 - DNA, 14 bp, Mouse
ttaaggaaatgaca

SEQ ID NO: 88 - DNA, 13 bp, Mouse
ttaaataaagaac

SEQ ID NO: 89 - DNA, 14 bp, Mouse
ttaatcccagtact

SEQ ID NO: 90 - DNA, 14 bp, Mouse
ttaaaataaacatg

SEQ ID NO: 91 - DNA, 14 bp, Mouse
ttaaaaatcaattt

SEQ ID NO: 92 - DNA, 14 bp, Mouse
ttaaaggtttttca

SEQ ID NO: 93 - DNA, 14 bp, Mouse
ttaaccaagatcaa

SEQ ID NO: 94 - DNA, 14 bp, Mouse
ttaagatctaaatt

SEQ ID NO: 95 - DNA, 14 bp, Mouse
ttaaggtgttttcc

SEQ ID NO: 96 - DNA, 14 bp, Mouse
ttaagataataatt

SEQ ID NO: 97 - DNA, 14 bp, Mouse
ttaaattcacgttg

SEQ ID NO: 98 - DNA, 14 bp, Mouse
ttaaactttaatct

SEQ ID NO: 99 - DNA, 14 bp, Mouse
ttaagaaacttaca

SEQ ID NO: 100 - DNA, 14 bp, Mouse
ttaaggcggaaatc

SEQ ID NO: 101 - DNA, 14 bp, Mouse
ttaaaaatattgtt

SEQ ID NO: 102 - DNA, 14 bp, Mouse
ttaataaaacatct

SEQ ID NO: 103 - DNA, 14 bp, Mouse
ttaaatttaccata

SEQ ID NO: 104 - DNA, 14 bp, Mouse
ttaaattttcctgg

SEQ ID NO: 105 - DNA, 14 bp, Mouse
ttaacaactgggat

SEQ ID NO: 106 - DNA, 14 bp, Mouse
ttaagagcttttta

SEQ ID NO: 107 - DNA, 14 bp, Mouse
ttaaatgaaaatta

SEQ ID NO: 108 - DNA, 14 bp, Mouse
ttaagaaaatgcct

SEQ ID NO: 109 - DNA, 14 bp, Mouse
ttaaaggtgctcat

SEQ ID NO: 110 - DNA, 14 bp, Mouse
ttaaaaatattaac

SEQ ID NO: 111 - DNA, 14 bp, Mouse
ttaagatggctaag

SEQ ID NO: 112 - DNA, 14 bp, Mouse
ttaagtaaaaaaaa

SEQ ID NO: 113 - DNA, 14 bp, Mouse
ttaacagtctattc

SEQ ID NO: 114 - DNA, 14 bp, Mouse
ttaagtagttaagc

SEQ ID NO: 115 - DNA, 13 bp, Mouse
ttaaggcacaata

SEQ ID NO: 116 - DNA, 14 bp, Mouse
ttaaagaaatcatc

SEQ ID NO: 117 - DNA, 14 bp, Mouse
ttaaaccaggatcc

SEQ ID NO: 118 - DNA, 14 bp, Mouse
ttaaaatacccttt

SEQ ID NO: 119 - DNA, 14 bp, Mouse
ttaatgaaaccttt

SEQ ID NO: 120 - DNA, 14 bp, Mouse
ttaaagaaggagag

SEQ ID NO: 121 - DNA, 14 bp, Mouse
ttaatcttatgtca

SEQ ID NO: 122 - DNA, 14 bp, Mouse
ttaagacctttcat

SEQ ID NO: 123 - DNA, 14 bp, Mouse
ttaacatactagat

SEQ ID NO: 124 - DNA, 14 bp, Mouse
ttaaaaaaatagat

SEQ ID NO: 125 - DNA, 14 bp, Mouse
ttaaaaaaaggaca

SEQ ID NO: 126 - DNA, 14 bp, Mouse
ttaaggagcattct

(19) United States
(12) Patent Application Publication
(10) Pub. No.: US 2010/0154070 A1
(43) Pub. Date: Jun. 17, 2010

(54) PiggyBac as a Tool for Genetic Manipulation and Analysis in Vertebrates

(76) Inventors: Tian Xu, Guilford, CT (US); Min Han, Boulder, CO (US); Yuan Zhuang, Chapel Hill, NC (US); Xiaohui Wu, Shanghai (CN); Sheng Ding, Shanghai (CN); Gang Li, Shanghai (CN)

Correspondence Address: JONES DAY, 222 EAST 41ST ST, NEW YORK, NY, 10017, US

(21) Appl. No.: 11/920,410

(22) PCT Filed: May 14, 2005
(86) PCT No.: PCT/CN2005/000674

§ 371 Date: Jan. 7, 2010

(51) Int. Cl. A01K 67/00; C12N 15/63; C12N 5/10; C07H 21/04; A01K 67/027

(52) U.S. Cl. 800/13; 435/455; 435/366; 536/23.1; 800/25

(57) ABSTRACT

The present invention relates to transgenic vertebrate, including mammalian, cells, whose genomes comprise one or more elements of the piggyBac family transposon system. Transgenic non-human vertebrates, including transgenic non-human mammals, whose genomes comprise one or more elements of the piggyBac family transposon system, are also provided. Methods of making and using the cells and animals of the invention, including applications in the medical, veterinary, and agricultural fields, are additionally provided. The present invention also relates to kits useful for practicing such methods.

### 1. FIELD OF THE INVENTION

The present invention relates to transgenic vertebrate, including mammalian, cells and transgenic non-human vertebrates, including non-human mammals, whose genomes comprise one or more elements of the piggyBac family transposon system, and methods of making and using the cells and animals. The present invention also relates to kits useful for practicing such methods.

### 2. BACKGROUND OF THE INVENTION

Transposable elements or transposons are mobile genetic units identified in many metazoa, including worms, insects, and humans. In humans and mice, transposon derived sequences account for more than 40% of the genome (Lander et al., 2001, Nature 409:860-921; Waterston et al., 2002, Nature 420:520-562), indicating the importance of transposition in evolution. Since the discovery of the first transposon in maize by McClintock (McClintock, 1950, Proc. Nat'l. Acad. Sci. USA 36:344-345), transposable elements have become invaluable tools for genetic analysis in many organisms. In prokaryotes, transposon based mutagenesis has led to discovery of genes important for microbial pathogenesis (Hutchison et al., 1999, Science 286:2165-2169; Vilen et al., 2003,1. Virol. 77:123-134). In eukaryotes, the introduction of P-element mediated transgenesis and insertional mutagenesis dramatically advanced Drosophila genetics (Rubin and Spradling, 1982, Science 218:348-353). Many transposons, including P-elements, are non-functional outside their natural hosts, suggesting host factors are involved in transposition (Handler et al., 1993, Archives of Insect Biochemistry & Physiology 22:373-384).

Transposon systems including members from the Tc1/Mariner family have been used in mouse and the zebrafish Danio rerio. By using a comparative phylogenetic approach, a synthetic Tel-like transposon Sleeping Beauty (SB) has been proven active in mouse and human cells (Ivies et al., 1997, Cell 91:501-510; Luo et al., 1998, Proc. Nat'l. Mad. Sci. USA 95:10769-10773). Although transposons such as Sleeping Beauty and Minos have been tested for insertional mutagenesis in mouse (Dupuy et al., 2001, Genesis 30:82-88; Fischer et al., 2001, Proc. Nat'l. Acad. Sci. USA 98:6759-6764; Horie et al., 2001, Proc. Nat'l. Acad. Sci, USA 98:9191-9196; Zagoraiou et al., 2001, Proc. Nat'l. Acad. Sci. USA 98:11474-11478), a general application of these transposons in mouse genetics is still limited due to the fact that new transposon insertions are heavily concentrated near the original site and occurred with low efficiencies (Drabek et al., 2003, Genomics 81:108-111; Dupuy et al., 2001, Genesis 30:82-88; Fischer et al., 2001, Proc. Nat'l. Acad. Sci. USA 98:6759-6764; Horie et al., 2001, Proc. Nat'l. Acad. Sci. USA 98:9191-9196; Horie et al., 2003, Mol. Cell Biol. 23:9189-9207; Zagoraiou et al., 2001, Proc. Nat'l. Acad. Sci. USA 98:11474-11478).

piggyBac elements are 2472-bp transposons with 13-bp inverted terminal repeats (“ITRs”) and a 594-amino acid transposase (Cary et al., Virology, Volume 161, 8-17, 1989). The piggyBac transposable element from the cabbage looper moth, Trichoplusia ni (Cary et al., Virology, Volume 161, 8-17, 1989) has been shown to be an effective gene-transfer vector in the Mediterranean fruit fly, Ceratitis capitata (Handler et al., Proc. Natl. Acad. Sci. USA, Volume 95, 7520-7525, 1998). Use of an unmodified transposase helper under piggyBac promoter regulation indicates that piggyBac retains autonomous function in the medfly, since transcriptional regulation was maintained, as well as enzymatic activity. This observation was unique since all other successful insect germline transformations had been limited to dipteran species using vectors isolated from the same or another dipteran. The initial transformation of medfly (Loukeris et al., Science, Volume 270, 2002-2005, 1995) used the Minos vector from Drosophila hydei (Franz & Savakis, Nucl. Acids Res., Volume 19, 6646, 1991), and Aedes aegypti has been transformed from Hermes (Jasinskiene et al., Proc. Natl. Acad. Sci. USA, Volume 95, 3743-3747, 1998) from Musca domestica (Warren et al., Genet. Res. Camb., Volume 64, 87-97, 1994) and mariner (Coates et al., Proc. Natl. Acad. Sci. USA, Volume 95, 3748-3751, 1998) from Drosophila mauritiana (Jacobson et al., Proc. Natl. Acad. Sci. USA, Volume 83, 8684-8688, 1986). Drosophila melanogaster has been transformed as well by Hermes (O'Brochta et al., Insect Biochem. Molec. Biol., Volume 26, 739-753, 1996), mariner (Lidholm et al., Genetics, Volume 134, 859-868, 1993), Minos (Franz et al., Proc. Natl. Acad. Sci. USA, Volume 91, 4746-4750, 1994) and by the P and hobo transposons originally discovered in its own genome (Rubin and Spradling, 1989; Blackman et al., EMBO J., Volume 8, 211-217, 1989). Drosophila virilis also has been transformed by hobo (Lozovskaya et al., Genetics, Volume 143, 365-374, 1995; Gomez & Handler, Insect Mol. Biol., Volume 6, 1-8, 1997) and mariner (Lohe et al., Genetics, Volume 143, 365-374, 1996). While the restriction to dipteran vectors is due in part to the limited number of transposon systems available from non-dipteran species, phylogenetic limitations on transposon function is not unexpected considering the deleterious effects functional transposons may have on a host genome. This is, indeed, reflected by the high level of regulation placed on transposon movement among species, among strains within a host species, and even among cell types within an organism (Berg & Howe, Mobile DNA, American Society for Microbiology, Washington, D.C. 1989).

piggyBac (PB) belongs to DNA transposons, elements of which generally excise from one genomic site and integrate into another by a cut-and-paste mechanism. It is a 2472-bp transposon with 13-bp inverted terminal repeats (ITRs) and a 594-amino acid transposase (Cary et al., 1989, Virology 172:156-169; Fraser et al., 1995, Virology 211:397-407; Fraser et al., 1996, Insect Molecular Biology 5:141-151). piggyBac elements have been used for genetic analysis in Drosophila melanogaster and other insects. It was found that the transposon inserted into the tetranucleotide TTAA site, which is duplicated upon insertion (Fraser et al., 1995, Virology 211:397-407; Fraser et al., 1996, Insect Molecular Biology 5:141-151). Because of the unique transposase and TTAA target site sequences, the transposon has been suggested as the founding member of a new DNA transposon family, the piggyBac family (Robertson, 2002, In Mobile DNA II, Craig et al., eds. (Washington, D.C., ASM Press), pp. 1093-1110). piggyBac has been used to transform the germline of more than a dozen species spanning four orders of insects (Handler, 2002, Insect Biochemistry & Molecular Biology 32:1211-1220; Sumitani et al., 2003, Insect Biochem. Mol. Biol. 33:449-458). As a mutagen, piggyBac transposes at least as effective as the P-element in Drosophila (Thibault et al., 2004, Nat. Genet. 36:283-287). In the red flour beetle Tribolium castaneum, piggyBac transposition also efficiently occurred between non-homologous chromosomes (Lorenzen et al., 2003, Insect Mol. Biol. 12:433-440). Many piggyBac-like sequences were found in the genomes of phylogenetically diverse species from fungi to mammals, further indicates that their activity may not be restricted to insects (Sarkar et al., 2003, Mol. Genet. Genomics 270:173-180). In fact, piggyBac has recently been shown capable of transposition in the planarian Girardia tigrina (Gonzalez-Estevez et al., 2003, Proc. Nat'l. Acad. Sci. USA 100:14046-14051).

Discussion or citation of a reference herein shall not be construed as an admission that such reference is prior art to the present invention.

### 3. SUMMARY OF THE INVENTION

The present invention is based on the surprising discovery that piggyBac can transpose efficiently in vertebrate, including mammalian, cells, both in vivo and ex vivo. piggyBac transposition occurs almost exclusively at TTAA sites following a precise cut-and-paste manner. When introduced into fertilized eggs, the piggyBac transposon could integrate into the mouse genome without obvious chromosome regional preferences, and preferably inserted into transcriptional units. Also, piggyBac elements can carry multiple marker genes and allow the expression of these genes at various insertion sites. Thus, the piggyBac transposon system, and other members of the “piggyBac-like” transposon family, are valuable new tools for efficient genetic manipulation and analysis in mice and other vertebrates.

The present invention provides methods of making transgenic non-human vertebrates comprising in the genomes of one or more of their cells a piggyBac-like transposon and/or a piggyBac-like transposase. Thus, methods of introducing piggyBac-like transposons and transposases into animals are provided herein, as are methods of mobilizing or immobilizing piggyBac-like transposons.

In certain embodiments, the present invention provides methods of generating a transgenic non-human vertebrate comprising in the genome of one or more of its cells a piggyBac-like transposon which carries an insert of at least 1.5 kb, comprising the steps of: (a) introducing ex vivo into a non-human vertebrate embryo or fertilized oocyte a nucleic acid comprising a piggyBac-like transposon which carries an insert of at least 1.5 kb and, within the same or on a separate nucleic acid, a nucleotide sequence encoding a piggyBac-like transposase; (b) implanting the resultant non-human vertebrate embryo or fertilized oocyte into a foster mother of the same species under conditions favoring development of said embryo into a transgenic non-human vertebrate; and (c) after a period of time sufficient to allow development of said embryo into a transgenic non-human vertebrate, recovering the transgenic non-human vertebrate from the mother; thereby generating a transgenic non-human vertebrate comprising in the genome of one or more of its cells piggyBac-like transposon which carries an insert of at least 1.5 kb.

As an alternative to introducing ex vivo into the non-human vertebrate embryo or fertilized oocyte a nucleic acid comprising a piggyBac-like transposon which carries an insert of at least 1.5 kb, a plurality of nucleic acids comprising overlapping portions of the piggyBac-like transposon can be introduced, as along as the overlap is sufficient for homologous recombination to take place inside the cell into which the nucleic acids are introduced. This alternative is particularly useful for introducing into the genome of a cell a piggyBac-like transposon that carries a large insert. Thus, in such embodiments, a first nucleic acid would harbor the left terminal of the piggyBac-like transposon and at least a portion of the insert and a second nucleic acid would harbor the right terminal of the piggyBac-like transposon and at least a portion of the insert. If only two nucleic acids are used, the portion of the insert harbored by the first nucleic acid and the portion of the insert harbored by the second nucleic acid overlap. If a third nucleic acid is used, the third nucleic acid would have regions of overlap with the first nucleic acid at one end and with the second nucleic acid at the other end. FIG. 14B illustrates such an embodiment. This principle of homologous recombination with multiple overlapping nucleic acids (e.g., two, three, four, five, six, or more) can be applied to introduce into the genomes of vertebrate cells and organisms piggyBac-like transposons with large inserts.

The present invention also provides a method of generating a transgenic non-human vertebrate comprising in the genome of one or more of its cells a piggyBac-like transposon which comprises a nucleotide sequence encoding a protein that modifies a trait in said transgenic non-human vertebrate, comprising the steps of (a) introducing ex vivo into a non-human vertebrate embryo or fertilized oocyte a nucleic acid comprising a piggyBac-like transposon which comprises a nucleotide sequence encoding a protein that modifies a trait in said transgenic non-human vertebrate, and, within the same or on a separate nucleic acid, a nucleotide sequence encoding a piggyBac-like transposase; (b) implanting the resultant non-human vertebrate embryo or fertilized oocyte into a foster mother of the same species under conditions favoring development of said embryo into a transgenic non-human vertebrate; and (c) after a period of time sufficient to allow development of said embryo into a transgenic non-human vertebrate, recovering the transgenic non-human vertebrate from the mother; thereby generating a transgenic non-human vertebrate comprising in the genome of one or more of its cells piggyBac-like transposon, said piggyBac-like transposon comprising a nucleotide sequence encoding a protein that modifies a trait in said transgenic non-human vertebrate.

The present invention further provides methods of generating a transgenic non-human vertebrate comprising in the genome of one or more of its cells a piggyBac-like transposon, wherein said piggyBac-like transposon is within a concatamer comprising a plurality of piggyBac-like transposons, said method comprising the steps of (a) introducing ex vivo into a non-human vertebrate embryo or fertilized oocyte a linearized nucleic acid comprising a piggyBac-like transposon and, within the same or on a separate nucleic acid, a nucleotide sequence encoding a piggyBac-like transposase; (b) implanting the resultant non-human vertebrate embryo or fertilized oocyte into a foster mother of the same species under conditions favoring development of said embryo into a transgenic non-human vertebrate; and (c) after a period of time sufficient to allow development of said embryo into a transgenic non-human vertebrate, recovering the transgenic non-human vertebrate from the mother, thereby generating a transgenic non-human vertebrate comprising in the genome of one or more of its cells a piggyBac-like transposon within a concatamer comprising a plurality of piggyBac-like transposons.

The present invention yet further provides methods of generating a transgenic non-human vertebrate comprising in the genome of one or more of its cells a nucleotide sequence encoding a piggyBac-like transposase, wherein said nucleotide sequence encoding the piggyBac-like transposase is within a concatamer comprising a plurality of nucleotide sequences, each of which encodes a piggyBac-like transposase, said method comprising the steps of: (a) introducing ex vivo into a non-human vertebrate embryo or fertilized oocyte a linearized nucleic acid comprising a nucleotide sequence encoding a piggyBac-like transposase; (b) implanting the resultant non-human vertebrate embryo or fertilized oocyte into a foster mother of the same species under conditions favoring development of said embryo into a transgenic non-human vertebrate; and (c) after a period of time sufficient to allow development of said embryo into a transgenic non-human vertebrate, recovering the transgenic non-human vertebrate from the mother, thereby generating a transgenic non-human vertebrate comprising in the genome of one or more of its cells a nucleotide sequence encoding a piggyBac-like transposase, wherein said nucleotide sequence encoding the piggyBac-like transposase is within a concatamer comprising a plurality of nucleotide sequences, each of which encodes a piggyBac-like transposase.

The present invention yet further provides methods of generating a transgenic non-human vertebrate comprising in the genome of one or more of its cells an immobilized piggyBac-like transposon, comprising the steps of: (a) introducing ex vivo into a non-human vertebrate embryo or fertilized oocyte (i) a nucleic acid comprising a piggyBac-like transposon; and (ii) piggyBac-like transposase polypeptide in an amount effective to induce the integration of said piggyBac-like transposon into the genome of one or more cells of said embryo or into the genome of said oocyte or one or more cells of an embryo derived therefrom, respectively; (b) implanting the resultant non-human vertebrate embryo or fertilized oocyte into a foster mother of the same species under conditions favoring development of said embryo into a transgenic non-human vertebrate; and (c) after a period of time sufficient to allow development of said embryo into a transgenic non-human vertebrate, recovering the transgenic non-human vertebrate from the mother; thereby generating a transgenic non-human vertebrate comprising in the genome of one or more of its cells an immobilized piggyBac-like transposon.

The present invention yet further provides methods of generating a recombinant vertebrate cell in culture whose genome comprises a piggyBac-like transposon which carries an insert of at least 1.5 kb, comprising the steps of: (a) introducing into a vertebrate cell in culture a nucleic acid comprising a piggyBac-like transposon which carries an insert of at least 1.5 kb, and, within the same or on a separate nucleic acid, a nucleotide sequence encoding a piggyBac-like transposase; and (b) culturing said cell under conditions in which the piggyBac-like transposase is expressed such the piggyBac-like transposon is integrated into the genome of said vertebrate cell in culture, thereby generating a recombinant vertebrate cell in culture whose genome comprises a piggyBac-like transposon which carries an insert of at least 1.5 kb.

As an alternative to introducing into a vertebrate cell in culture a nucleic acid comprising a piggyBac-like transposon which carries an insert of at least 1.5 kb, a plurality of nucleic acids comprising overlapping portions of the piggyBac-like transposon can be introduced, as along as the overlap is sufficient for homologous recombination to take place inside the cell into which the nucleic acids are introduced. As described above, by using multiple nucleic acids comprising only portions piggyBac-like transposons and their inserts, this alternative is particularly useful for generating and introducing into the genome of a cell a piggyBac-like transposon that carries a large insert.

The present invention yet further provides methods of generating a recombinant vertebrate cell in culture whose genome comprises a piggyBac-like transposon which comprises a nucleotide sequence encoding a protein of value in the treatment or prevention of a vertebrate disease or disorder, comprising the steps of (a) introducing into a vertebrate cell in culture a nucleic acid comprising a piggyBac-like transposon which comprises a nucleotide sequence encoding a protein of value in the treatment or prevention of a vertebrate disease or disorder, and, within the same or on a separate nucleic acid, a nucleotide sequence encoding a piggyBac-like transposase; (b) culturing said cell under conditions in which the piggyBac-like transposase is expressed such the piggyBac-like transposon is integrated into the genome of said vertebrate cell in culture, thereby generating a recombinant vertebrate cell in culture whose genome comprises a piggyBac-like transposon which comprises a nucleotide sequence encoding a protein of value in the treatment or prevention of a vertebrate disease or disorder.

The present invention yet further provides methods of generating a recombinant vertebrate cell in culture whose genome comprises a piggyBac-like transposon, wherein said piggyBac-like transposon is within a concatamer comprising a plurality of piggyBac-like transposons, comprising the steps of: (a) introducing into a vertebrate cell in culture a linearized nucleic acid comprising a piggyBac-like transposon, and, within the same or on a separate nucleic acid, a nucleotide sequence encoding a piggyBac-like transposase; (b) culturing said cell under conditions in which the piggyBac-like transposase is expressed such the piggyBac-like transposon is integrated into the genome of said vertebrate cell in culture, thereby generating a recombinant vertebrate cell in culture whose genome comprises a piggyBac-like transposon, wherein said piggyBac-like transposon is within a concatamer comprising a plurality of piggyBac-like transposons.

The present invention yet further provides methods of generating a recombinant vertebrate cell in culture whose genome comprises a nucleotide sequence encoding a piggyBac-like transposase, wherein said nucleotide sequence encoding a piggyBac-like transposase is within a concatamer comprising a plurality of nucleotide sequences, each of which encodes a piggyBac-like transposase, comprising the steps of: (a) introducing into a vertebrate cell in culture a linearized nucleic acid comprising a nucleotide sequence encoding a piggyBac-like transposase, and (b) culturing said cell under conditions in which the nucleotide sequence encoding a piggyBac-like transposase is integrated into the genome of said vertebrate cell in culture, thereby generating a recombinant vertebrate cell in culture whose genome comprises a nucleotide sequence encoding a piggyBac-like transposase, wherein said nucleotide sequence encoding said piggyBac-like transposase is within a concatamer comprising a plurality of nucleotide sequences, each of which encodes a piggyBac-like transposase.

The present invention yet further provides methods of mobilizing a piggyBac-like transposon in a non-human vertebrate, comprising the steps of: (a) mating a first transgenic non-human vertebrate comprising in the genome of one or more of its germ cells a piggyBac-like transposon, wherein said piggyBac-like transposon carries an insert of at least 1.5 kb, with a second transgenic non-human vertebrate comprising in the genome of one or more of its germ cells a nucleotide sequence encoding a piggyBac-like transposase to yield one or more progeny; (b) identifying at least one of said one or more progeny of step (a) comprising in the genome of one or more of its cells both said piggyBac-like transposon and said nucleotide sequence encoding the piggyBac-like transposase, such that the piggyBac-like transposase is expressed and the transposon is mobilized; thereby mobilizing the piggyBac-like transposon in a non-human vertebrate. The first and second transgenic non-human vertebrates can be generated according to any of the methods described herein.

The present invention yet further provides methods of mobilizing a piggyBac-like transposon in a non-human vertebrate, comprising the steps of: (a) mating a first transgenic non-human vertebrate comprising in the genome of one or more of its germ cells a piggyBac-like transposon, wherein said piggyBac-like transposon comprises a nucleotide sequence encoding a protein that modifies a trait in said transgenic non-human vertebrate, with a second transgenic non-human vertebrate comprising in the genome of one or more of its germ cells a nucleotide sequence encoding a piggyBac-like transposase to yield one or more progeny; (b) identifying at least one of said one or more progeny of step (a) comprising in the genome of one or more of its cells both said piggyBac-like transposon and said nucleotide sequence encoding the piggyBac-like transposase, such that the piggyBac-like transposase is expressed and the transposon is mobilized; thereby mobilizing the piggyBac-like transposon in a non-human vertebrate. The first and second transgenic non-human vertebrates can be generated according to any of the methods described herein.

The present invention yet further provides methods of mobilizing a piggyBac-like transposon in a non-human vertebrate, comprising the steps of (a) mating a first transgenic non-human vertebrate comprising in the genome of one or more of its germ cells a piggyBac-like transposon, wherein said piggyBac-like transposon is within a concatamer comprising a plurality of piggyBac-like transposons, with a second transgenic non-human vertebrate comprising in the genome of one or more of its germ cells a nucleotide sequence encoding a piggyBac-like transposase to yield one or more progeny; (b) identifying at least one of said one or more progeny of step (a) comprising in the genome of one or more of its cells both said piggyBac-like transposon and said nucleotide sequence encoding the piggyBac-like transposase, such that the piggyBac-like transposase is expressed and the transposon is mobilized; thereby mobilizing the piggyBac-like transposon in a non-human vertebrate. The first and second transgenic non-human vertebrates can be generated according to any of the methods described herein.

The present invention yet further provides methods of immobilizing a piggyBac-like transposon in a non-human vertebrate, comprising the steps of: (a) mating a first transgenic non-human vertebrate comprising in the genome of one or more of its cells both (i) a piggyBac-like transposon which comprises an insert of at least 2 kb and (ii) a nucleotide sequence encoding a piggyBac-like transposase with a second adult vertebrate to yield one or more progeny; (b) identifying at least one of said one or more progeny of step (a) that does not comprise in its genome the nucleotide sequence encoding the piggyBac-like transposase, and comprises in the genome of one or more of its cells a piggyBac-like transposon, such that the piggyBac-like transposon is immobilized in said progeny, thereby immobilizing the piggyBac-like transposon in a non-human vertebrate. The first transgenic non-human vertebrate can be generated according to any of the methods described herein. The second transgenic non-human vertebrate is not necessarily a transgenic animal; however, if is transgenic, then it can be generated according to any of the methods described herein.

The present invention yet further provides methods of immobilizing a piggyBac-like transposon in a non-human vertebrate, comprising the steps of: (a) mating a first transgenic non-human vertebrate comprising in the genome of one or more of its cells both (i) a piggyBac-like transposon which comprises a nucleotide sequence encoding a protein that modifies a trait in said transgenic non-human vertebrate and (ii) a nucleotide sequence encoding a piggyBac-like transposase with a second adult vertebrate to yield one or more progeny; (b) identifying at least one of said one or more progeny of step (a) that does not comprise in its genome the nucleotide sequence encoding the piggyBac-like transposase, and comprises in the genome of one or more of its cells a piggyBac-like transposon, such that the piggyBac-like transposon is immobilized in said progeny, thereby immobilizing the piggyBac-like transposon in a non-human vertebrate. The first transgenic non-human vertebrate can be generated according to any of the methods described herein. The second transgenic non-human vertebrate is not necessarily a transgenic animal; however, if is transgenic, then it can be generated according to any of the methods described herein.

The present invention yet further provides methods of immobilizing a piggyBac-like transposon in a non-human vertebrate, comprising the steps of: (a) mating a first transgenic non-human vertebrate comprising in the genome of one or more of its cells both (i) a piggyBac-like transposon, wherein said piggyBac-like transposon is within a concatamer comprising a plurality of piggyBac-like transposons, and (ii) a nucleotide sequence encoding a piggyBac-like transposase with a second adult vertebrate to yield one or more progeny; (b) identifying at least one of said one or more progeny of step (a) that does not comprise in its genome the nucleotide sequence encoding the piggyBac-like transposase, and comprises in the genome of one or more of its cells a piggyBac-like transposon, such that the piggyBac-like transposon is immobilized in said progeny, thereby immobilizing the piggyBac-like transposon in a non-human vertebrate. The first transgenic non-human vertebrate can be generated according to any of the methods described herein. The second transgenic non-human vertebrate is not necessarily a transgenic animal; however, if is transgenic, then it can be generated according to any of the methods described herein.

The present invention yet further provides methods of generating a transgenic non-human vertebrate which comprises in the genome of one or more of its cells an immobilized piggyBac-like transposon, said method comprising the steps of: (a) generating a transgenic non-human vertebrate comprising in the genome of a plurality of its germline cells both (i) a piggyBac-like transposon and (ii) a nucleotide sequence encoding a piggyBac-like transposase operably linked to a promoter that is expressed in the germline, wherein at least one of said piggyBac-like transposon and said nucleotide sequence encoding the piggyBac-like transposase is within a concatamer comprising a plurality of piggyBac-like transposons or a concatamer comprising a plurality of nucleotide sequences each of which encodes a piggyBac-like transposase, comprising the steps of: introducing ex vivo into a non-human vertebrate embryo or fertilized oocyte one or more nucleic acids, said one or more nucleic acids comprising (i) a piggyBac-like transposon and (ii) a nucleotide sequence encoding a piggyBac-like transposase linked to a promoter that is expressed in the germline, wherein at least one of said one or more nucleic acids is linearized; implanting the resultant non-human vertebrate embryo or fertilized oocyte into a foster mother of the same species under conditions favoring development of said embryo into a transgenic non-human vertebrate; and after a period of time sufficient to allow development of said embryo into a transgenic non-human vertebrate, recovering a transgenic non-human vertebrate from the mother that comprises in the genome of a plurality of its germline cells both (i) a piggyBac-like transposon and (ii) a nucleotide sequence encoding a piggyBac-like transposase operably linked to a promoter that is expressed in the germline, wherein at least one of said piggyBac-like transposon and said nucleotide sequence encoding the piggyBac-like transposase is within a concatamer comprising a plurality of piggyBac-like transposons or a concatamer comprising a plurality of nucleotide sequences each of which encodes a piggyBac-like transposase; (b) allowing the recovered transgenic non-human vertebrate of step (a) to grow into adulthood; (c) mating the adult transgenic non-human vertebrate of step (b) with a second adult vertebrate to yield one or more progeny; (d) identifying at least one of said one or more progeny of step (c) that does not comprise in its genome the nucleotide sequence encoding the piggyBac-like transposase operably linked to the promoter that is expressed in the germline, and comprises in the genome of one or more of its cells a piggyBac-like transposon, wherein said one or more progeny is each a transgenic non-human vertebrate which comprises in the genome of one or more of its cells an immobilized piggyBac-like transposon; thereby generating a transgenic non-human vertebrate which comprises in the genome of one or more of its cells an immobilized piggyBac-like transposon.

The present invention yet further provides methods of generating a library of transgenic non-human vertebrates, each of which comprises in the genome of one or more of its cells an immobilized piggyBac-like transposon, said method comprising the steps of: (a) generating a transgenic non-human vertebrate comprising in the genome of a plurality of its germline cells both (i) a piggyBac-like transposon and (ii) a nucleotide sequence encoding a piggyBac-like transposase operably linked to a promoter that is expressed in the germline, wherein at least one of said piggyBac-like transposon and said nucleotide sequence encoding the piggyBac-like transposase is within a concatamer comprising a plurality of piggyBac-like transposons or a concatamer comprising a plurality of nucleotide sequences each of which encodes a piggyBac-like transposase, comprising the steps of: introducing ex vivo into a non-human vertebrate embryo or fertilized oocyte one or more nucleic acids, said one or more nucleic acids comprising (i) a piggyBac-like transposon and (ii) a nucleotide sequence encoding a piggyBac-like transposase linked to a promoter that is expressed in the germline, wherein at least one of said one or more nucleic acids is linearized; implanting the resultant non-human vertebrate embryo or fertilized oocyte into a foster mother of the same species under conditions favoring development of said embryo into a transgenic non-human vertebrate; and after a period of time sufficient to allow development of said embryo into a transgenic non-human vertebrate, recovering a transgenic non-human vertebrate from the mother that comprises in the genome of a plurality of its germline cells both (i) a piggyBac-like transposon and (ii) a nucleotide sequence encoding a piggyBac-like transposase operably linked to a promoter that is expressed in the germline, wherein at least one of said piggyBac-like transposon and said nucleotide sequence encoding the piggyBac-like transposase is within a concatamer comprising a plurality of piggyBac-like transposons or a concatamer comprising a plurality of nucleotide sequences each of which encodes a piggyBac-like transposase; (b) allowing the recovered transgenic non-human vertebrate of step (a) to grow into adulthood; (c) mating the adult transgenic non-human vertebrate of step (b) with a second adult vertebrate to yield a plurality of progeny; (d) identifying two or more progeny of step (c), each of which does not comprise in its genome the nucleotide sequence encoding the piggyBac-like transposase operably linked to the promoter that is expressed in the germline, and comprises in the genome of one or more of its cells a piggyBac-like transposon, wherein said two or more progeny is each a transgenic non-human vertebrate which comprises in the genome of one or more of its cells an immobilized piggyBac-like transposon, thereby generating a library of transgenic non-human vertebrates, each comprising in the genome of one or more of its cells an immobilized piggyBac-like transposon.

In certain aspects, the present invention further provides a transgenic non-human vertebrate, comprising in the genome of one or more of its cells a piggyBac-like transposon and/or a piggyBac-like transposase. In certain embodiments, the transposon carries an insert of at least 1.5 kb; comprises a nucleotide sequence encoding a protein that modifies a trait in said transgenic non-human vertebrate; and/or is within a concatamer comprising a plurality of piggyBac-like transposons.

In certain aspects, the present invention further provides a vertebrate cell in culture comprising in its genome a piggyBac-like transposon and/or a piggyBac-like transposase. In certain embodiments, the transposon carries an insert of at least 1.5 kb; comprises a nucleotide sequence encoding a protein that modifies a trait in a transgenic non-human vertebrate; is within a concatamer comprising a plurality of piggyBac-like transposons; and/or comprises a nucleotide sequence encoding a protein of value in the treatment or prevention of a vertebrate disease or disorder.

The present invention further provides libraries of the transgenic non-human vertebrates or vertebrate cells in culture described herein. In certain embodiments, the libraries are produced by the methods of the invention. In certain embodiments, a library of transgenic non-human vertebrates comprises at least 6, at least 10, at least 20, at least 50 or at least 100 members, at least some, or preferably all, of which harbor a piggyBac-like transposon at a different position in the genome. A library vertebrate cells in culture, in certain embodiments, comprises at least 10, at least 20, at least 50, at least 100 members, or at least 1000 members, at least some, or preferably all, of which harbor a piggyBac-like transposon at a different position in the genome. Thus, in certain embodiments, the present invention provides libraries of transgenic non-human vertebrates or vertebrate cells in culture, the genomes of which harbor piggyBac-like transposons, wherein the transpsons carry an insert of at least 1.5 kb; comprise a nucleotide sequence encoding a protein that modifies a trait in a transgenic non-human vertebrate; are within a concatamer comprising a plurality of piggyBac-like transposons; and/or comprise a nucleotide sequence encoding a protein of value in the treatment or prevention of a vertebrate disease or disorder.

The methods and composotions of the invention are useful in treating or preventing diseases and disorders. Thus, in certain aspects, the present invention provides methods of treating or preventing a disease or disorder, said method comprising the step of administering a recombinant vertebrate cell whose genome comprises a piggyBac-like transposon which comprises a nucleotide sequence encoding a protein of value in the treatment or prevention of the vertebrate disease or disorder to a subject in need of such treatment or prevention.

In other aspects, the present invention provides methods of delivering a nucleic acid encoding a protein of value in the treatment or prevention of a vertebrate disorder to one or more cells of a subject in need of such treatment or prevention, said method comprising the step of administering a recombinant virus whose genome comprises (i) a piggyBac-like transposon which comprises a nucleotide sequence encoding said protein and (ii) a nucleotide sequence encoding a piggyBac-like transposase operably linked to a promoter that directs expression of the piggyBac-like transposase in said one or more cells of said subject, such that the piggyBac-like transposon is integrated into the genome of said one or more cells of said subject following said administration, thereby delivering a nucleic acid encoding a protein of value in the treatment or prevention of a vertebrate disorder to a subject in need of such treatment or prevention. In certain embodiments, the virus can be a retrovirus, an adenovirus, or an adeno-associated virus.

The present invention further provides a recombinant virus, e.g., a retrovirus, an adenovirus, or an adeno-associated virus, whose genome comprises (i) a piggyBac-like transposon which comprises a nucleotide sequence encoding said protein and (ii) a nucleotide sequence encoding a piggyBac-like transposase operably linked to a promoter.

Because of the precise excision of piggyBac-like transposons, the present methods can be useful for determining whether a phenotype exhibited by a transgenic non-human vertebrate comprising in the genome of one or more of its cells a piggyBac-like transposon is caused by the piggyBac-like transposon. In certain aspects, said methods comprise the steps of: (a) generating one or more progeny of said transgenic non-human vertebrate in which the piggyBac-like transposon is excised; (b) determining whether a correlation exists between the excision of said piggyBac-like transposon in said progeny and a reversion of the phenotype, wherein a correlation is indicative that the phenotype is caused by the piggyBac-like transposon, thereby determining whether a phenotype exhibited by a transgenic non-human vertebrate comprising in the genome of one or more of its cells a piggyBac-like transposon is caused by the piggyBac-like transposon.

The piggyBac-like transposons of the invention are useful in enhancer trapping. Thus, the present invention provides methods for isolating an enhancer from a non-human vertebrate or from a vertebrate cell in culture. In certain aspects, the methods comprise the steps of: (a) assessing in a transgenic non-human vertebrate comprising in the genome of one or more of its cells or tissues a piggyBac-like transposon, wherein the transposon comprises a reporter gene under the control of a minimal promoter, the expression of the reporter gene in said one or more cells or tissues of the transgenic non-human vertebrate or offspring derived therefrom; and (b) isolating a nucleic acid flanking said piggyBac-like transposon that is responsible for the expression of the reporter gene in said one or more cells or tissues; thereby isolating an enhancer from a non-human vertebrate. In other aspects, the methods, useful for isolating an enhancer from a recombinant vertebrate cell in culture, wherein the recombinant cell comprises piggyBac-like transposon comprising a reporter gene under the control of a minimal promoter, comprise the steps of: (a) assessing the expression of the reporter gene in said recombinant vertebrate cell or its progeny; and (b) isolating a nucleic acid flanking said piggyBac-like transpon that is responsible for the expression of the reporter gene in recombinant vertebrate cell; thereby isolating an enhancer from a recombinant vertebrate cell in culture.

The methods of the invention are useful method for generating chimeric non-human vertebrate animals. Thus, in certain aspects, the present invention provides methods of generating transgenic non-human vertebrates whose cells are mosaic for a piggyBac-like transposon, comprising the steps of: (a) generating a transgenic non-human embryo comprising within its genome (i) a genetic locus homozygous for a piggyBac-like transposon, wherein the piggyBac-like transposon comprises a site-specific recombinase recognition sequence, and (ii) a nucleotide sequence encoding said site-specific recombinase operably linked to a promoter; (b) culturing the transgenic non-human embryo under conditions in which the site-specific recombinase is expressed and proliferation occurs; thereby generating a non-human transgenic vertebrate that whose cells are mosaic for a piggyBac-like transposon. Chimeric animals produced by such methods are also encompassed by the present invention.

The present invention further provides kits comprising materials suitable for practicing the invention. Thus, in certain aspects, the invention provides kits comprising (a) in one or more containers, one or more nucleic acids comprising (i) a piggyBac-like transposon and (ii) a nucleotide sequence encoding a piggyBac-like transposase; and (b) in a second container, (i) a vertebrate cell in culture or (ii) a non-human vertebrate oocyte. In specific embodiments, the piggyBac-like transposon carries an insert of at least 1.5 kb and/or carries an insert encoding a protein of value in the treatment or prevention of a vertebrate disease or disorder. In certain aspects, at least one nucleic acid in a kit of the invention is linearized.

In certain embodiments of the methods and compositions claimed herein, the piggyBac-like transposon which comprises a nucleotide sequence encoding a protein that modifies a trait in said transgenic non-human vertebrate.

In certain aspects of the methods and compositions claimed herein, the nucleic acid comprising the piggyBac-like transposon is linearized, such that the genome of one or more of said cells comprises said piggyBac-like transposon within a concatamer, said comprising a plurality of piggyBac-like transposons.

In yet other aspects of the methods and compositions claimed herein, the nucleic acid comprising the nucleotide sequence encoding the piggyBac-like transposase is linearized, such that the genome of one or more of said cells comprises said nucleotide sequence encoding the piggyBac-like transposase within a concatamer, the concatamer comprising a plurality of nucleotide sequences each of which encodes a piggyBac-like transposase.

In yet other aspects of the methods and compositions claimed herein, the piggyBac-like transposon comprises a sequence recognized by a protein that binds to and/or modifies nucleic acids. In certain embodiments, the nucleic acid-modifying protein is a DNA-binding protein, a DNA-modifying protein, an RNA-binding protein, or an RNA-modifying protein. The nucleic acid-modifying protein can also be a target site for a site-specific recombinase, for example a target site for FRT or lox recombinase.

In yet other aspects of the methods and compositions of the invention, the piggyBac-like transposon comprises a selectable marker. In yet other aspects, the piggyBac-like transposon comprises a reporter gene. In a specific embodiment, the piggyBac-like transposon comprises both a selectable marker and a reporter gene. In another specific embodiment, the reporter gene is endogenous to the species to which the transposon is introduced.

In yet other aspects of the methods and compositions of the invention, the piggyBac-like transposon comprises an insert of at least 0.5 kb, at least 1 kb, or at least 1.5 kb. In other embodiments, the piggyBac-like transposon comprises an insert of at least 2 kb, at least 2.5 kb, at least 3 kb, at least 4 kb, at least 5 kb, at least 6 kb, at least 7 kb, at least 8 kb, at least 9 kb, at least 10 kb, at least 11 kb, at least 11.5 kb, at least 13 kb, at least 14 kb, or at least 15 kb. In other specific embodiments, the piggyBac-like transposon comprises an insert no greater than 15 kb, no greater than 20 kb, no greater than 25 kb, no greater than 30 kb, no greater than 35 kb, no greater than 40 kb, no greater than 45 kb, no greater than 50 kb, no greater than 60 kb, no greater than 75 kb, or no greater than 100 kb. In yet other specific embodiments, the piggyBac-like transposon comprises an insert of ranging between 1.5-3 kb, 1.5-5 kb, 1.5-10 kb, 1.5-20 kb, 1.5-30 kb, 1.5-50 kb, 1.5-75 kb, 2-5 kb, 2-10 kb, 2-20 kb, 2-30 kb, 2-50 kb, 2-75 kb, 3-5 kb, 3-10 kb, 3-20 kb, 3-30 kb, 3-50 kb, 3-75 kb, 5-10 kb, 5-20 kb, 5-30 kb, 5-50 kb, 5-75 kb, 10-20 kb, 10-30 kb, 10-50 kb, or 10-75 kb.

Where the methods or compositions of the invention entail the introduction of both a piggyBac-like transposon and a nucleotide sequence encoding a piggyBac transposase into a cell or organism, the piggyBac-like transposon and the nucleotide sequence encoding the piggyBac-like transposase can be within the same nucleic acid or on separate nucleic acids. In an embodiment where the transposon and the transposase coding region are on separate nucleic acids, the nucleic acid comprising the piggyBac-like transposon is DNA and the nucleic acid comprising the piggyBac-like transposase is RNA, allowing the piggyBac-like transposon to be immobilized in the genome of said cell or organism.

Alternatively, the nucleic acids comprising the piggyBac-like transposon and the piggyBac-like transposase can both be DNA, allowing the generation of a cell or organism whose genome comprises a nucleotide sequence encoding a piggyBac-like transposase. Preferably, the nucleotide sequence encoding the piggyBac-like transposase is operably linked to a promoter. In one embodiment, the promoter directs expression of the transposase in the germline, for example is a ubiquitous promoter or, more preferably, is a germline-specific promoter. In one embodiment, the germline specific promoter is a male-specific promoter (e.g., Protamine 1 (Prm) promoter, as described herein). In another embodiment, the germline specific promoter is a female-specific promoter (e.g., a ZP3 promoter).

The subjects of the therapeutic and prophylactic methods of the invention are preferably non-human vertebrate. In preferred embodiments, the subject is a human or non-human animal. In specific embodiments, the animal is a pet (e.g., cat, dog) or a livestock (cow, horse) animal.

In certain embodiments, the transgenic non-human vertebrate of the invention is a bird (e.g., chicken or other fowl), or fish (e.g., zebrafish). In other embodiments, the vertebrate is a non-human mammal, including but not limited to non-human primate, cow, cat, dog, horse, sheep, mouse, rat, guinea pig, panda, and pig. In a specific embodiment, the transgenic non-human vertebrate is a livestock animal.

The recombinant cell of the invention can be any vertebrate cell. In specific embodiments, the cell is of avian (e.g., chicken or other fowl) or fish (e.g., zebrafish) origin. In other embodiments, the cell is of mammalian origin, including but not limited to primate (including but not limited to human cells and chimpanzee cells), cow, cat, dog, horse, sheep, mouse, rat, guinea pig, hamster, mink, panda, and pig. In other embodiments, the cell is a frog cell, e.g., a Xenopus laevis cell. In a specific embodiment, the cell's origin is of a livestock animal. The cell can be normal or diseased, and of any differentiation type or state.

In certain embodiments of the present invention, the nucleic acids harboring the piggyBac-like transposon and/or transposase coding-sequence are linearized prior to their introduction into a cell or organism, such that the nucleic acid is inserted to the genome of said or organism as a concatamer.

Preferably, the piggyBac-like transposon employed in the methods and compositions of the invention is a piggyBac transposon, and/or the piggyBac-like transposase is a piggyBac transposase.

The present invention also provides embodiments covering any and all permutations of the features described herein. All values and ranges in between all the values listed herein, for example with respect to piggyBac-like transposon insert size or cell/organism library size, are also encompassed by the present invention.

### 4. BRIEF DESCRIPTION OF THE DRAWINGS

FIG. 1. Transposon vectors and transposase constructs of the piggyBac binary transposon system for mammalian cells and mice. (FIG. 1A) PB donor constructs. Marker or endogenous genes (shaded boxes with arrows denoting transcription direction) driven by various promoters were placed between a pair of PB repeat termini (PBL and PBR, black arrows). Arrowheads above the termini show the relative positions of primers used for inverse PCR. Total lengths of the transposons are also indicated. Open boxes represent the plasmid backbone sequences. M: MfeI; B: BamHI; S: SwaI; A: AscI; H: HindIII. (FIG. 1B) PB transposase helper constructs. The piggyBac transposase gene (PBase) driven by cytomegalovirus (CMV), beta-actin (Act), or Protamine 1 (Prm1) promoters were followed by either bovine growth hormone polyA (BGH pA) or rabbit beta-globin polyA (rBG pA).

FIG. 2. piggyBac integration in mammalian cultured cells. (FIG. 2A) Statistical results of enhanced transgene integration in 293 cells. The numbers of G418-resistant clones were scored from transfections of donor transposon construct with or without helper plasmids. Each number is the average obtained from three transfection experiments. The bar shows the standard deviation (P<0.0001). (FIG. 2B) Statistical results of enhanced transgene integration in mouse W4/129S6 ES cells. Clones were counted as in (A). (FIG. 2C) An example of mouse ES cell transfection experiments. Surviving clones were stained with methylene blue after G418 selection.

FIG. 3. piggyBac elements transposition in mice (FIG. 3A) Ratio of the transposon-positive founders determined by PCR genotyping among all pups resulting from injection of circular plasmids. The solid bars and open bars represent the results from co-injections of the donor and helper plasmids or injections of the donor plasmid alone, respectively. The presence of the PB transposase resulted in an elevated transgenic efficiency. (FIG. 3B) Southern analysis of PB[Act-RFP] positive founders. In some cases more than 10 integrations in a single founder mouse (AP0-41) were observed, while no signals were found in the wild-type control. (FIG. 3C) Southern analysis indicated germline transmission of PB elements. After mating with wild-type animals, founders and their progenies were analyzed. Multiple PB[Act-RFP] integrations in a male founder (AF0-61) were segregated in its offspring. A female PB[Act-RFP] founder (AF0-47) that carried a single PB[Act-RFP] transposition integration (judged by the Southern and the inverse PCR result, A47T6 in Table 3) also transmitted its transposon to one of its progeny (47-336).

FIG. 4. Precise excision and transposition of PiggyBac in mouse germline. A male founder mouse co-injected with Prm1-PBase and PB[Act-RFP] was used for analyzing germline transposition. (FIG. 4A) Scaled structure of the PB[Act-RFP] transposon. Genomic DNA is represented by curved lines, while the PB transposon-containing plasmid concatamer is shown in aligned boxes. Restriction sites: M: MluIE: EcoRVB: BglII A: Acc65I. Position of the probe for Southern analysis is illustrated by the solid line. Primers used to detect excision events are shown as arrowheads. (FIG. 4B) Southern analysis of a founder (BF0-33) and its progeny revealed bands other than the 1.3 kb concatamer signal, thus implying the occurrence of germline transposition. (FIG. 4C) Positive bands with expected length from precise excision were observed in several progenies after PCR amplification with the primers shown in (FIG. 4A).

FIG. 5. Expression of transgenes in piggyBac vectors (FIG. 5A) PB[Act-RFP] expression in the progenies resulted in red fluorescence under the illumination of a portable long-wave UV light. Two positive mice carrying the same single copy transposon (arrows) and two negative littermates (stars) are shown. (FIG. 5B) PB[Act-RFP] expression in a founder mouse and her progeny. Red fluorescence was mosaic in the founder. Segregation of transposons in the progeny resulted in different intensities of RFP signal. The asterisk marks the transgene-negative littermate. (FIG. 5C) and (FIG. 5D) Co-expression of two transgenes in the same piggyBac vector. As a result of tyrosinase expression, a PB[K14-Tyr, Act-RFP] founder shows grey coat color under white light, while the transgene-negative littermate remains albino (FIG. 5C, right and left, respectively). When illuminated by UV, red fluorescence was observed from this founder (FIG. 5D).

FIG. 6. piggyBac integration sites in mouse (FIG. 6A) Nucleotide composition of flanking sequences from 100 PB integration sites. In addition to the TTAA target site specificity, an enrichment of Ts and As in the flanking sequences was observed. Asterisks denote P<0.05 when compared with flanking sequence of the randomly sampled TTAA control. (FIG. 6B) Distribution of PB insertions in genes. Percentages of the PB insertions located in exons, introns, 5′ regulatory sequences (10 kb adjacent to transcription start site), 3′ regulatory sequences (10 kb adjacent to polyA site), and in all four regions (total) are illustrated. Solid bars indicate data from all known and predicted genes and empty bars indicated data from the known genes or ESTs. (FIG. 6C) Distribution of PB insertions in 5′ regions. (FIG. 6D) Distribution of PB insertions in 3′ regions. (FIG. 6E) Analysis of 93 integration sites in mice showed that PB integrations appeared to hit all but the two smallest chromosomes (19 and Y). Filled arrowheads indicate hits in exons, dark arrowheads indicate hits in introns, empty arrowheads indicate hits in predicted intergenic regions.

FIG. 7. Enhanced piggyBac integration in various mammalian cell lines.

FIG. 8 piggyBac can transpose in different species.

FIG. 9. piggyBac can excise and transpose in mouse somatic cells. Mice doubly positive for the Act-PBase and a concatomer of PB was obtained by crossing. PCR with the PB flanking primers detected the excisions of the transposons from their original sites. New transpon insertions were further revealed by inverse PCR from the same individual. These events were not detected in their PB single positive parents. Since the DNA were extracted from the tail sample, the excisions and transpositions are expected to happen in somatic cells.

FIG. 10. Transposition by coinjection of piggyBac transposon with the Pmr-piggyBac transposase construct.

FIG. 11A-C. Transposition by crossing. FIG. 11A. A male germline specific promoter (prm) was further tested with the crossing strategy. Mice carrying piggyBac transposon were crossed with mice carrying the Pmr-PBase transgene, the results showing that a crossing strategy can be utilized to induce new transpositions. FIG. 11B. Mice doubly positive for the Act-PBase and a concatomer of PB were crossed with wild type mice. New transposition events were detected in the progenies of this cross by inverse PCR and Southern blot. All three new transpositions tested could be stably transmitted to the next generation. FIG. 11C. A male mouse doubly positive for Pmr-PBase and a concatemer of PB (DF1-9) actively produced progeny carrying new transposon insertions (as revealed by Southern in the left panel and inverse PCR). About 50% of the new insertions were located near to the putative original site on chromosome four, which suggests that local hopping could happen when PB jumps. The mice carrying the non-autonomous PB transposons (concatemer or single copy) were crossed with the mice carrying the transposase (eg. Pmr-PBase which expresses the transposase specifically in the mouse germline). Transposon/transposase doubly positive F1 mice (only male mice in the case of Pmr-Pbase), were crossed with widetype mice. In the next generation (F2), Southern blot and inverse PCR were used to clone the new transposition sites. Using both Pmr-Pbase and Act-Pbase, new insertions were obtain in every recombinations tried, even when mice carrying a single copy of transposon were used as a starter line. One of the transpositions analyzed originated in chromosome 5 and landed in chromosome 1.

FIG. 12A-B. piggyBac insertions report gene expression patterns. lacZ-containing piggyBac transposons report the expression patterns of the genes into which they are inserted, Two Examples Insertions in F27iR43 (FIG. 12A) and in Grb10 (FIG. 12B). In FIG. 12B, the results of a PB-based exon trap vector carrying a lacZ reporter gene are shown. When the transposon inserted into the first intron of Grb10, lacZ staining of the mouse embryos shown the expression pattern of Grb10 compatible to results reported by others.

FIG. 13A-B. piggyBac insertions can cause phenotypes in mice. Two Examples: Insertions in Pkd2 gene cause embryonic lethality (recessive, causing focal hemorrhage and whole-body edema in Pkd2 homozygous embryos) (FIG. 13A-B) and in Eya1 gene cause eye defect (dominant) (FIG. 13B), just like mutant mice generated by traditional knockout methods.

FIG. 14A-B illustrates the use of the piggyBac-like transposon system to insert large pieces of DNA into vertebrate genomes. FIG. 14A shows a plasmid, a cosmid, a P1 fragment, or a BAC fragment that carries one or more genes (represented by dark arrows) and is cloned into the inverted terminal repeats (ITRs) of a piggyBac-like transposon. In the presence of a piggyBac-like transposase (circles), the whole cassette would be integrated into the genome (solid line) by transposition. Alternatively, as shown in FIG. 14B, a large chromosome region is cut into several partial overlapping fragments, with two most outward pieces each carrying a piggyBac-like ITR. In the presence of piggyBac-like transposase, these fragments would be integrated into the genome (solid line) by transposition and homologous recombination.

### 5. DETAILED DESCRIPTION OF THE INVENTION

The present invention provides applications of piggyBac-like transposon systems in vertebrate cells and non-human vertebrate organisms. The invention provides vertebrate cells and non-human organisms engineered to express components of the piggyBac-like transposon system, methods of making such cells and organisms, libraries of such engineered cells and organisms.

The invention relates to the introduction of the piggyBac-like transposon of the invention to the genome of a cell. Efficient incorporation of the transposon occurs when the cell also contains a piggyBac-like transposase. As discussed above, the piggyBac-like transposase can be provided to the cell as piggyBac-like transposase protein or as nucleic acid encoding the piggyBac-like transposase. Nucleic acid encoding the piggyBac-like transposase can take the form of RNA or DNA. Further, the nucleic acid encoding the piggyBac-like transposase can be stably or transiently incorporated into the cell to facilitate temporary or prolonged expression of the piggyBac-like transposase in the cell. Further, promoters or other expression control regions can be operably linked with the nucleic acid encoding the piggyBac-like transposase to regulate expression of the protein in a quantitative or in a tissue-specific manner.

The piggyBac-like transposon of this invention can introduced into one or more cells using any of a variety of techniques known in the art such as, but not limited to, microinjection, combining a nucleic acid comprising the transposon with lipid vesicles, such as cationic lipid vesicles, particle bombardment, electroporation, DNA condensing reagents (e.g., calcium phosphate, polylysine or polyethyleneimine) or incorporating the transposon into a viral vector and contacting the viral vector with the cell. Where a viral vector is used, the viral vector can include any of a variety of viral vectors known in the art including viral vectors selected from the group consisting of a retroviral vector, an adenovirus vector or an adeno-associated viral vector.

The piggyBac-like transposon system of this invention can readily be used to produce transgenic animals that carry a particular marker or express a particular protein in one or more cells of the animal. Methods for producing transgenic animals are known in the art.

In another application of this invention, the invention provides a method for mobilizing a piggyBac-like sequence in a cell. In this method the piggyBac-like transposon is incorporated into DNA in a cell. Additional piggyBac-like transposase or nucleic acid encoding the piggyBac-like transposase is introduced into the cell and the protein is able to mobilize (i.e., move) the nucleic acid fragment from a first position within the DNA of the cell to a second position within the DNA of the cell. The method permits the movement of the nucleic acid fragment from one location in the genome to another location in the genome, or for example, from a plasmid in a cell to the genome of that cell. In one embodiment, the cell is in culture.

Mobilization of piggyBac-like transposons can also take place in the context of an animal, for example by mating two adults, one of which harbors the piggyBac-like transposon in at least some of its germ cells and another that harbors a piggyBac-like transposase coding sequence in at least some of its germ cells, thereby generating progeny that harbor both transposon and transposase. Alternatively, a transgenic animal is generated by co-injecting nucleic acids for a piggyBac-like transposon and transposase (on the same or separate nucleic acids) into an ovum or fertilized egg, thereby generating a transgenic animal comprising both a piggyBac-like transposon and transposase coding sequence. The transposase coding sequence can be placed under a ubiquitous or a tissue-specific promoter, so that it is expressed in at least some cells that harbor the transposon. This allows for the mobilization of the transposon. If the promoter is active in the germline, then the progeny of the animal may inherit the mobilized transposon. To ensure the stability of the mobilized transposon, progeny are selected that do not comprise the transposase-encoding gene. In such progeny, the transposon is immobilized.

The piggyBac-like transposon systems of the invention comprising piggyBac-like transposons in combination with the piggyBac-like transposase protein or nucleic acid encoding the piggyBac-like transposase are powerful tools for germline transformation, for the production of transgenic animals, for the introduction of nucleic acid into DNA into a cell, for insertional mutagenesis, and for gene tagging in a variety of vertebrate species.

The invention further provides applications of this system in vertebrates as a tool for efficient genetic manipulation and analysis, with applications in the medical, pharmaceutical and livestock industries.

5.1. PiggyBac-Like Transposon Systems

The present invention relates to the use of piggyBac-like transposon systems in vertebrate cells. Such systems are used to introduce nucleic acid sequences into the DNA of a vertebrate cell. The piggyBac-like transposases bind to recognition sites in the inverted repeats of the piggyBac-like transposons and catalyze the incorporation of the transposon into DNA, such as the genomic DNA of a target cell. As illustrated in the examples, the combination of the piggyBac-like transposon and the piggyBac-like transposase-encoding nucleic acid of this results in the integration of the transposon sequence into a cell or organism.

piggyBac-like transposons are mobile, in that they can move from one position on DNA to a second position on DNA in the presence of a piggyBac-like transposase. There are two fundamental components of the piggyBac-like transposon system, a source of an active piggyBac-like transposase and the piggyBac-like ITRs that are recognized and mobilized by the transposase. Mobilization of the ITRs permits the intervening nucleic acid between the ITRs to also be mobilized.

The piggyBac-like transposon system of this invention, therefore, comprises two components: a piggyBac-like transposase or nucleic encoding a piggyBac-like transposase, and a cloned piggyBac-like transposon, which is a nucleic acid comprising at least two inverted repeats recognized by a piggyBac-like transposase). When put together these two components provide active transposon activity. In use, the transposase binds to the inverted repeats and promotes integration of the intervening nucleic acid sequence into DNA of a cell.

The practice of the methods of the composition thus involves a bi-partite piggyBac-like transposon system, comprising a piggyBac-like transposon element and a piggyBac-like transposase or a nucleic acid encoding a piggyBac-like transposase. The piggyBac-like components can be derived from piggyBac or any related piggyBac-like transposon system.

In the piggyBac-like transposons of the present invention, the left and right transposon terminals (which contain the 5′ and 3′ terminal inverted repeats recognized by a piggyBac-like transposase) flank an insert, for example a nucleic acid that is to be inserted into a target cell genome or encodes a selectable or phenotypic marker, as described in greater detail below.

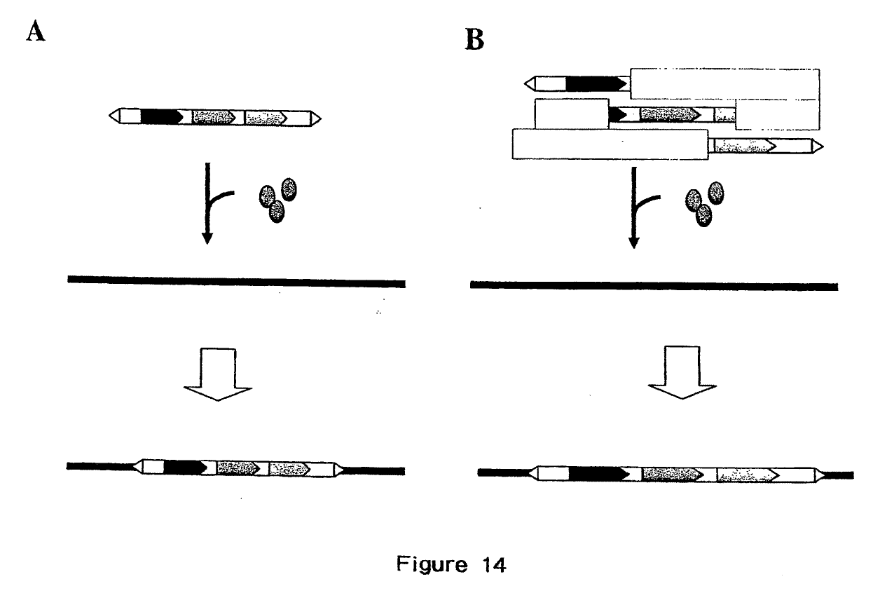

The insert located or positioned between the left and right terminals of the piggyBac-like transposon may vary greatly in size. Indeed, the inventors have made the suprising discovery that piggyBaccan stably transpose even when carrying large inserts of 14 kb or more. In specific embodiments, the insert is at least 0.5 kb, at least 1 kb, at least 1.5 kb, at least 2 kb, at least 2.5 kb, at least 3 kb, at least 4 kb, at least 5 kb, at least 6 kb, at least 7 kb, at least 8 kb, at least 9 kb, at least 10 kb, at least 11 kb, at least 11.5 kb, at least 13 kb, at least 14 kb, or at least 15 kb. In other specific embodiments, the piggyBac-like transposon comprises an insert no greater than 15 kb, no greater than 20 kb, no greater than 25 kb, no greater than 30 kb, no greater than 35 kb, no greater than 40 kb, no greater than 45 kb, no greater than 50 kb, no greater than 60 kb, no greater than 75 kb, or no greater than 100 kb. In yet other specific embodiments, the piggyBac-like transposon comprises an insert of ranging between 1.5-3 kb, 1.5-5 kb, 1.5-10 kb, 1.5-20 kb, 1.5-30 kb, 1.5-50 kb, 1.5-75 kb, 2-5 kb, 2-10 kb, 2-20 kb, 2-30 kb, 2-50 kb, 2-75 kb, 2.5-5 kb, 2.5-10 kb, 2.5-20 kb, 2.5-30 kb, 2.5-50 kb, 2.5-75 kb, 3-5 kb, 3-10 kb, 3-20 kb, 3-30 kb, 3-50 kb, 3-75 kb, 5-10 kb, 5-20 kb, 5-30 kb, 5-50 kb, 5-75 kb, 10-20 kb, 10-30 kb, 10-50 kb, or 10-75 kb.

Where the insert is of a size that is sufficiently large so as to inactivate the ability of the transposon system to integrate the transposon into the target genome, the transposon can be supplied in overlapping portions (e.g., two or three or four) on different nucleic acids, such that homologous recombination would allow the different nucleic acids to recombine within the cell and integrate into the genome as a single, large transposon in the presence of a piggyBac-like transposase. Thus, in such embodiments, a first nucleic acid would harbor the left terminal of the piggyBac-like transposon and at least a portion of the insert and a second nucleic acid would harbor the right terminal of the piggyBac-like transposon and at least a portion of the insert. If only two nucleic acids are used, the portion of the insert harbored by the first nucleic acid and the portion of the insert harbored by the second nucleic acid overlap. If a third nucleic acid is used, the third nucleic acid would have regions of overlap with the first nucleic acid at one end and with the second nucleic acid at the other end. FIG. 14B illustrates such an embodiment. This principle of homologous recombination with multiple overlapping nucleic acids (e.g., two, three, four, five, six, or more) can be applied to introduce into the genomes of vertebrate cells and organisms piggyBac-like transposons with large inserts. In this manner, transposons with inserts of up to 50 kb, 60 kb, 75 kb, 100 kb, 120 kb, 140 kb, 160 kb or even more can be introduced into the genome of a target cell.

This homologous recombination system advantageously allows the insertion of large pieces of DNA into target cells, for example entire genes comprising introns, exons and regulatory elements. The extent of overlap between each pair of nucleic acids will depend on the recombination requirements for the target cell, but can be as little as about 20 nucleotides to several kilobases. In specific embodiments, the extent of overlap is at least 50 nucleotides, at least 100 nucleotides, at least 200 nucleotides, at least 300 nucleotides, at least 500 nucleotides, at least 750 nucleotides or at least 1 kb. In other embodiments, the extent of overlap is no greater than 750 nucleotides, no greater than 1 kb, no greater than 1.5 kb or no greater than 1.5 kb.

As mentioned above, the piggyBac-like transposon system of the present invention also includes a source of piggyBac-like transposase activity. The piggyBac-like transposase activity is one that binds to the inverted repeats of the piggyBac-like transposon and mediates integration of the transposon into the genome of the target cell. Any suitable piggyBac-like transposase activity may be employed in the subject methods so long as it meets the above parameters. The piggyBac-like transposase activity can be from the same source or from a difference source as the piggyBac-like transposon itself.

The source of piggyBac-like transposase activity may vary. In certain embodiments, the source may be a protein that exhibits piggyBac-like transposase activity. However, the source is generally a nucleic acid that encodes a protein having piggyBac-like transposase activity. Where the source is a nucleic acid which encodes a protein having piggyBac-like transposase activity, the nucleic acid encoding the transposase protein is generally part of an expression module, as described above, where the additional elements provide for expression of the transposase as required. The transposase can thus be integrated into the genome of a target cell. However, in certain embodiments, the transposase is provided to the cell as a protein or as an RNA.

The piggyBac-like transposon of the present invention is generally introduced into a target cell on a vector, such as a plasmid, a viral-based vector, a linear DNA molecule, and the like. Preferably, the piggyBac-like transposon comprises an insert containing at least a portion of an open reading frame. Suitable open reading frames are provided in Section 5.14. In one embodiment the piggyBac-like transposon insert further contains a regulatory region, such as a transcriptional regulatory region (e.g., a promoter, an enhancer, a silencer, a locus-control region, or a border element). Suitable regulatory regions are provided in Section 5.11. Preferably, the regulatory region is linked to the open reading frame.

In certain embodiments where the source of transposase activity is a nucleic acid encoding a piggyBac-like transposase, the piggyBac-like transposon and the nucleic acid encoding the transposase are present on separate vectors, e.g., separate plasmids. In certain other embodiments, the transposase encoding sequence may be present on the same vector as the transposon, e.g., on the same plasmid. When present on the same vector, the piggyBac-like transposase encoding region or domain is located outside the transposon ITRs.

Exemplary transposon systems from which the transposon and transposase elements of the invention may be obtained are listed in Table 1, below:

TABLE 1
suitable sources of transposon systems

| Name or description | Reference | Suitable source of |
|---|---|---|
| pk[BIG-alpha] piggyBac | Genbank Accession No. AF402295 | Transposon |
| transformation vector | | |
| PiggyBac helper plasmid | Genbank Accession No. AY196821 | Transposase |
| pBlu-uTp, complete sequence | | |
| Phytophthora infestans | Genbank Accession No. AY830111 | Transposon |
| PiggyBac-like transposon | | Transposase |
| PiggyPi-1 | | |
| PiggyBac transformation | Genbank Accession No. AY196822 | Transposon |
| vector pB-MCS w+ | | |
| PiggyBac transformation | Genbank Accession No. AY196823 | Transposon |
| vector pB-UAS w+ | | |
| PiggyBac transformation | Genbank Accession No. AY196824 | Transposon |
| vector pB-UGateway w+ | | |
| PiggyBac transformation | Genbank Accession No. AY196825 | Transposon |
| vector pB-UGIR w+ | | |
| PiggyBac ubiquitin- | Genbank Accession No. AY196826 | Transposase |
| transposase P replacement | | |
| vector EP3005 | | |
| Cloning vector piggyBac_PB | Genbank Accession No. AY515146 | Transposon |
| Cloning vector piggyBac_RB | Genbank Accession No. AY515147 | Transposon |
| Cloning vector piggyBac_WH | Genbank Accession No. AY515148 | Transposon |
| Heliothis virescens transposon | Genbank Accession No. AY264805 | Transposase |
| piggyBac transposase gene | | |
| More than 50 piggyBac-like | Sarkar et al., 2003, Mol. Genet. | Transposon |
| sequences | Genomics 270(2): 173-80. | Transposase |
| piggyBac-like sequences in | Kapitonov & Jurka, 2003, Proc Natl | Transposon |
| Drosophila melanogaster | Acad Sci USA 100(11): 6569-74. | Transposase |
| piggyBac-like sequences from | Robertson, 2002, In Mobile DNA II, | Transposon |
| a variety of species | Craig et al., eds. (Washington, D.C., | Transposase |
| | ASM Press), pp. 1093-1110 | |

In addition to the specific piggyBac-like transposase sequences provided in Table 1 above, and in Section 6 below, the piggyBac-like transposase may be encoded by DNA that can hybridize to a transposase-encoding nucleic acid provided in Table 1 under stringent hybridization conditions, as long as the encoded protein retains transposase activity with respect to a piggyBac-like transposon. In specific embodiments, the transposase is encoded by a nucleotide sequence with at least 60%, 70%, 80%, 90%, 95%, 98% or 99% sequence identity to the piggyBac-like transposase-encoding sequences provided in Table 1.

In certain embodiments, there are a variety of conservative changes that can be made to the amino acid sequence of the piggyBac-like transposase without altering piggyBac-like activity. These changes are termed conservative mutations, that is, an amino acid belonging to a grouping of amino acids having a particular size or characteristic can be substituted for another amino acid, particularly in regions of the protein that are not associated with catalytic activity or DNA binding activity, for example. Other amino acid sequences of the piggyBac-like transposase include transposes with amino acid sequences containing conservative changes relative to the sequences presented herein that do not significantly alter the function of the transposase. Substitutes for an amino acid sequence may be selected from other members of the class to which the amino acid belongs. For example, the nonpolar (hydrophobic) amino acids include alanine, leucine, isoleucine, valine, proline, phenylalanine, and tryptophan. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Particularly preferred conservative substitutions include, but are not limited to, Lys for Arg and vice versa to maintain a positive charge; Glu for Asp and vice versa to maintain a negative charge; Ser for Thr so that a free hydroxyl group is maintained; and Gln for Asn to maintain a free amino group.

Further, a particular DNA sequence encoding a piggyBac-like transposase can be modified to employ the codons preferred for a particular cell type, e.g., the codons for the target cell into which the transposase coding sequence is to be introduced.

In addition to the piggyBac-like transposon sequences specifically enumerated in Table 1, and in Section 6 below, the term “piggyBac-like transposon” encompasses any DNA fragments that could be excised by natural or artificial transposases and reinserted into a TTAA target site in the genome, causing a target-site duplication (TSD) that flanks the element. In specific embodiments, this sequence is derived from piggyBac or a piggyBac-like element listed in Table 1 or described in Section 6 below.

5.2. Methods of Preparing the piggyBac-Like Transposon System

The various elements of the piggyBac-like transposon system employed in the subject methods, e.g., vectors comprising the piggyBac-like transposon or transposase elements, may be produced by standard methods of restriction enzyme cleavage, ligation and molecular cloning. One protocol for constructing the subject vectors includes the following steps. First, purified nucleic acid fragments containing desired component nucleotide sequences as well as extraneous sequences are cleaved with restriction endonucleases from initial sources, e.g., a vector comprising the piggyBac-like transposon. Fragments containing the desired nucleotide sequences are then separated from unwanted fragments of different size using conventional separation methods, e.g., by agarose gel electrophoresis. The desired fragments are excised from the gel and ligated together in the appropriate configuration so that a circular nucleic acid or plasmid containing the desired sequences as described herein is produced. Where desired, the circular molecules so constructed are then amplified in a prokaryotic host, e.g., E. coli. The procedures of cleavage, plasmid construction, cell transformation and plasmid production involved in these steps are well known to one skilled in the art and the enzymes required for restriction and ligation are available commercially (see, e.g., T. Maniatis, E. F. Fritsch and J. Sambrook, Molecular Cloning: A Laboratory Manual, Cold Spring Harbor Laboratory Press, Cold Spring Harbor, N.Y. (1982); Catalog 1982-83, New England Biolabs, Inc.; Catalog 1982-83, Bethesda Research Laboratories, Inc.). Additional examples of how to construct the vectors employed in the subject methods is provided in Section 6, infra.

5.3. Using the piggyBac-Like Transposon System to Integrate a Nucleic Acid into a Target Cell Genome

The methods described herein find use in a variety of applications in which it is desired to introduce and stably integrate an exogenous nucleic acid into the genome of a target cell or organism.

Organisms of interest include vertebrates, where the vertebrate is a mammal in many embodiments. In certain embodiments, the vertebrate of the invention is a bird (e.g., chicken or other fowl), or fish (e.g., zebrafish). In other embodiments, the vertebrate is a non-human mammal, including but not limited to non-human primate, cow, cat, dog, horse, sheep, mouse, rat, hamster, mink, guinea pig, panda, and pig. In other embodiments, the oganism is a frog, e.g., a Xenopus laevis. In a specific embodiment, the transgenic non-human vertebrate is a livestock animal.

In embodiments involving administration of the transposon system directly to the multicellular organism, for example for gene therapy purposes (described more extensively in Section 5.4, infra) the mammal can also be a human.

5.4. Methods of Introducing the PiggyBac-Like Transposition System Into Multicellular Organisms

The route of the piggyBac-like transposon system to a multicellular organism depends on several parameters, including: the nature of the vectors that carry the system components, the nature of the delivery vehicle, the nature of the organism, and the like.

A common feature of this mode of administration is that it provides for in vivo delivery of the transposon system components to the target cell(s). In certain embodiments, linear or circularized DNA, e.g., a plasmid, is employed as the vector for delivery of the transposon system to the target cell. In such embodiments, the plasmid may be administered in an aqueous delivery vehicle, e.g., a saline solution. Alternatively, an agent that modulates the distribution of the vector in the multicellular organism may be employed. For example, where the vectors comprising the subject system components are plasmid vectors, lipid based, e.g., liposome, vehicles may be employed, where the lipid based vehicle may be targeted to a specific cell type for cell or tissue specific delivery of the vector. Patents disclosing such methods include: U.S. Pat. Nos. 5,877,302; 5,840,710; 5,830,430; and 5,827,703, the disclosures of which are herein incorporated by reference. Alternatively, polylysine based peptides may be employed as carriers, which may or may not be modified with targeting moieties, and the like. (Brooks, A. I., et al. 1998, J. Neurosci. Methods V. 80 p: 137-47; Muramatsu, T., Nakamura, A., and H. M. Park 1998, Int. J. Mol. Med. V. 1 p: 55-62). In yet other embodiments, the system components may be incorporated onto viral vectors, such as adenovirus derived vectors, sindbis virus derived vectors, retroviral derived vectors, etc. hybrid vectors, and the like. The above vectors and delivery vehicles are merely representative. Any vector/delivery vehicle combination may be employed, so long as it provides for in vivo administration of the transposon system to the multicellular organism and target cell. Suitable vector/delivery vehicles in the gene therapy context are provided in Section 5.13 below.

Because of the multitude of different types of vectors and delivery vehicles that may be employed, administration may be by a number of different routes, where representative routes of administration include: oral, topical, intraarterial, intravenous, intraperitoneal, intramuscular, etc. The particular mode of administration depends, at least in part, on the nature of the delivery vehicle employed for the vectors which harbor the piggyBac-like transposon system. In many embodiments, the vector or vectors harboring the piggyBac-like transposon system are administered intravascularly, e.g., intraarterially or intravenously, employing an aqueous based delivery vehicle, e.g., a saline solution.

The elements of the piggyBac-like transposon system, e.g., the piggyBac-like transposon and the piggyBac-like transposase source, are administered to the multicellular organism in an in vivo manner such that they are introduced into a target cell of the multicellular organism under conditions sufficient for excision of the inverted repeat flanked nucleic acid from the vector carrying the transposon and subsequent integration of the excised nucleic acid into the genome of the target cell. Depending on the structure of the transposon vector itself, i.e., whether or not the vector includes a region encoding a product having piggyBac-like transposase activity, the method may further include introducing a second vector into the target cell which encodes the requisite transposase activity.

The amount of vector nucleic acid comprising the transposon element, and in many embodiments the amount of vector nucleic acid encoding the transposase, that is introduced into the cell is sufficient to provide for the desired excision and insertion of the transposon nucleic acid into the target cell genome. As such, the amount of vector nucleic acid introduced should provide for a sufficient amount of transposase activity and a sufficient copy number of the nucleic acid that is desired to be inserted into the target cell. The amount of vector nucleic acid that is introduced into the target cell varies depending on the efficiency of the particular introduction protocol that is employed, e.g., the particular in vivo administration protocol that is employed.

The particular dosage of each component of the system that is administered to the multicellular organism varies depending on the nature of the transposon nucleic acid, e.g., the nature of the expression module and gene, the nature of the vector on which the component elements are present, the nature of the delivery vehicle and the like. Dosages can readily be determined empirically by those of skill in the art. For example, in mice where the piggyBac-like transposon system components are present on separate plasmids which are intravenously administered to a mammal in a saline solution vehicle, the amount of transposon plasmid that is administered in many embodiments typically ranges from about 0.5 to 40 and is typically about 25 μg, while the amount of piggyBac-like transposase encoding plasmid that is administered typically ranges from about 0.5 to 25 and is usually about 1 μg.

Once the vector DNA has entered the target cell in combination with the requisite transposase, the nucleic acid region of the vector that is flanked by inverted repeats, i.e., the vector nucleic acid positioned between the piggyBac-like transposase recognized inverted repeats, is excised from the vector via the provided transposase and inserted into the genome of the targeted cell. As such, introduction of the vector DNA into the target cell is followed by subsequent transposase mediated excision and insertion of the exogenous nucleic acid carried by the vector into the genome of the targeted cell.

The subject methods may be used to integrate nucleic acids of various sizes into the target cell genome, as described in Section 5.1, supra.

The subject methods result in stable integration of the nucleic acid into the target cell genome. By stable integration is meant that the nucleic acid remains present in the target cell genome for more than a transient period of time, and is passed on a part of the chromosomal genetic material to the progeny of the target cell.

5.5. Methods of Generating Recombinant Cells Comprising PiggyBac-Like Transposons

The creation of a transformed cell requires that the DNA first be physically placed within the host cell. Current transformation procedures utilize a variety of techniques to introduce DNA into a cell. In one form of transformation, the DNA is microinjected directly into cells though the use of micropipettes. Alternatively, high velocity ballistics can be used to propel small DNA associated particles into the cell. In another form, the cell is permeabilized by the presence of polyethylene glycol, thus allowing DNA to enter the cell through diffusion. DNA can also be introduced into a cell by fusing protoplasts with other entities which contain DNA. These entities include minicells, cells, lysosomes or other fusible lipid-surfaced bodies. Electroporation is also an accepted method for introducing DNA into a cell. In this technique, cells are subject to electrical impulses of high field strength which reversibly permeabilizes biomembranes, allowing the entry of exogenous DNA sequences. One preferred method of introducing the transformation construct into cells in accordance with the present invention is to microinject fertilized eggs with the construct. The DNA sequence flanked by the transposon inverted repeats will be inserted into the genome of the fertilized egg during development of the organism, this DNA will be passed on to all of the progeny cells to produce a transgenic organism. The microinjection of eggs to produce transgenic animals has been previously described and utilized to produce transformed mammals (Hogan et al., Manipulating The Mouse Embryo: A Laboratory Manual, Cold Spring Harbor Laboratory Press, Plainview, N.Y., 1986; Shirk et al., In Biotechnology For Crop Protection, Hedin et al (eds.), ACS Books, Washington D.C., 135-146, 1988; Morgan et al., Annu. Rev., Biochem., Volume 62, 191-217, 1993; all herein incorporated by reference).

Alternatively, the two part piggyBac-like transposon system can be delivered to cells via viruses, including retroviruses (including lentiviruses), adenoviruses, adeno-associated viruses, herpesviruses, and others. There are several potential combinations of delivery mechanisms for the transposon portion containing the transgene of interest flanked by the inverted terminal repeats (ITRs) and the gene encoding the transposase. For example, both the transposon and the transposase gene can be contained together on the same recombinant viral genome; a single infection delivers both parts of the piggyBac-like system such that expression of the transposase then directs cleavage of the transposon from the recombinant viral genome for subsequent integration into a cellular chromosome. In another example, the transposase and the transposon can be delivered separately by a combination of viruses and/or non-viral systems such as lipid-containing reagents. In these cases either the transposon and/or the transposase gene can be delivered by a recombinant virus. In every case, the expressed transposase gene directs liberation of the transposon from its carrier DNA (viral genome) for integration into chromosomal DNA.

The piggyBac-like transposon systems of the invention may be introduced into any cell line or primary cell line of vertrebrate origin.

In certain embodiments, the cell is of a cell line, such as Chinese hamster ovary (CHO), HeLa, VERO, BHK, Cos, MDCK, 293, 3T3, myeloma (e.g. NSO, NSI), HT-1080, or W138 cells. The vertebrate cell can also be the product of a cell fusion event, such as a hybridoma cell.

In certain embodiments, the cell can be a pluripotent cell (i.e., a cell whose descendants can differentiate into several restricted cell types, such as hematopoietic stem cells or other stem cells) or a totipotent cell (i.e., a cell whose descendants can become any cell type in an organism, e.g., embryonic stem cells). Cells such as oocytes, eggs, and one or more cells of an embryo are also considered in this invention.

In yet other embodiments, the cells can be mature cells, from a variety of organs or tissues. Such cells include, but are not limited to, lymphocytes, hepatocytes, neural cells, muscle cells, a variety of blood cells, and a variety of cells of an organism.

5.6. Methods of Generating Recombinant Animals Comprising PiggyBac-Like Transposons

The piggyBac-like transgenes described above are introduced into nonhuman mammals. Most nonhuman mammals, including rodents such as mice and rats, rabbits, ovines such as sheep and goats, porcines such as pigs, and bovines such as cattle and buffalo, are suitable.

In some methods of transgenesis, transgenes are introduced into the pronuclei of fertilized oocytes. For some animals, such as mice fertilization is performed in vivo and fertilized ova are surgically removed. In other animals, particularly bovines, it is preferably to remove ova from live or slaughterhouse animals and fertilize the ova in vitro. See DeBoer et al., WO 91/08216. In vitro fertilization permits a transgene to be introduced into substantially synchronous cells at an optimal phase of the cell cycle for integration (not later than S-phase). Transgenes are usually introduced by microinjection. See U.S. Pat. No. 4,873,292. Fertilized oocytes are then cultured in vitro until a pre-implantation embryo is obtained containing about 16-150 cells. The 16-32 cell stage of an embryo is described as a morula. Pre-implantation embryos containing more than 32 cells are termed blastocysts. These embryos show the development of a blastocoel cavity, typically at the 64 cell stage. Methods for culturing fertilized oocytes to the pre-implantation stage are described by Gordon et al. (1984) Methods Enzymol. 101, 414; Hogan at al., Manipulation of the Mouse Embryo: A Laboratory Manual, C.S.H.L. N.Y. (1986) (mouse embryo); and Hammer et al. (1985) Nature 315, 680 (rabbit and porcine embryos); Gandolfi et al. (1987) J. Reprod. Fert. 81, 23-28; Rexroad et al. (1988) J. Anim. Sci. 66, 947-953 (ovine embryos) and Eyestone et al., (1989) J. Reprod. Fert. 85, 715-720; Camous et al. (1984) J. Reprod. Fert. 72, 779-785; and Heyman et al. (1987) Theriogenology 27, 5968 (bovine embryos) (incorporated by reference in their entirety for all purposes). Sometimes pre-implantation embryos are stored frozen for a period pending implantation. Pre-implantation embryos are transferred to an appropriate female resulting in the birth of a transgenic or chimeric animal depending upon the stage of development when the transgene is integrated. Chimeric mammals can be bred to form true germline transgenic animals.

Alternatively, transgenes can be introduced into embryonic stem cells (ES). These cells are obtained from preimplantation embryos cultured in vitro. Bradley et al. (1984), Nature 309, 255-258 (incorporated by reference in its entirety for all purposes). Transgenes can be introduced into such cells by electroporation or microinjection. Transformed ES cells are combined with blastocysts from a nonhuman animal. The ES cells colonize the embryo and in some embryos form the germ line of the resulting chimeric animal. See Jaenisch, Science, 240, 1468-1474 (1988) (incorporated by reference in its entirety for all purposes). Alternatively, ES cells can be used as a source of nuclei for transplantation into an enucleated fertilized oocyte giving rise to a transgenic mammal.

For production of transgenic animals containing two or more transgenes, e.g., in embodiments where the piggyBac-like transposon and piggyBac-like transposase components of the invention are introduced into an animal via separate nucleic acids, the transgenes can be introduced simultaneously using the same procedure as for a single transgene. Alternatively, the transgenes can be initially introduced into separate animals and then combined into the same genome by breeding the animals. Alternatively, a first transgenic animal is produced containing one of the transgenes. A second transgene is then introduced into fertilized ova or embryonic stem cells from that animal.

In some embodiments, transgenes whose length would otherwise exceed about 50 kb, are constructed as overlapping fragments. Such overlapping fragments are introduced into a fertilized oocyte or embryonic stem cell simultaneously and undergo homologous recombination in vivo. See Kay et al., WO 92/03917 (incorporated by reference in its entirety for all purposes).

Transgenic mammals can be generated conventionally by introducing by microinjecting the above-described transgenes into mammals' fertilized eggs (those at the pronucleus phase), implanting the eggs in the oviducts of female mammals (recipient mammals) after a few additional incubation or directly in their uteri synchronized to the pseudopregnancy, and obtaining the youngs.

To find whether the generated youngs are transgenic, below-described dot-blotting, PCR, immunohistological, complement-inhibition analyses and the like can be used.

The transgenic mammals thus generated can be propagated by conventionally mating and obtaining the youngs, or transferring nuclei (nucleus transfer) of the transgenic mammal's somatic cells, which have been initialized or not, into fertilized eggs of which nuclei have previously been enucleated, implanting the eggs in the oviducts or uteri of the recipient mammals, and obtaining the clone youngs.

Transformed cells and/or transgenic organisms (those containing the DNA inserted into the host cell's DNA) can be selected from untransformed cells and/or transformed organisms if a selectable marker was included as part of the introduced DNA sequences. Selectable markers include, for example, genes that provide antibiotic resistance; genes that modify the physiology of the host, such as for example green fluorescent protein, to produce an altered visible phenotype; etc. Cells and/or organisms containing these genes are capable of surviving in the presence of antibiotic, insecticides or herbicide concentrations that kill untransformed cells/organisms or producing an altered visible phenotype. Using standard techniques known to those familiar with the field, techniques such as, for example, Southern blotting and polymerase chain reaction, DNA can be isolated from transgenic cells and/or organisms to confirm that the introduced DNA has been inserted.

5.7. PiggyBac-Like Transposons Carrying Site-Specific Recombinase Recognition Sites

The piggyBac-like transposon system of the invention can be used to insert site-specific recombinase recognition sequences randomly in the chromosome of non-human vertebrates to facilitate generation of mutant and/or mosaic animals. In specific embodiments, the site-specific recombinase is the Cre-loxP system or the FLP-FRT system (see Kilby, 1993, Trends Genet 9(12):413-421 and references cited therein).

Recombination between two site-specific recombinase recognition sequences integrated on different chromosomes results in translocation between those chromosomes. Such translocations are a common means of creating mutations that lead to developmental abnormalities or tumorigenesis.

Recombination between two site-specific recombinase recognition sequences in direct repeat orientation may cause excision of an intervening DNA sequence (e.g., a gene). Although such events are potentially reversible, loss of the excised DNA sequence during cell division or by degradation makes the mutation irreversible. A null mutation in any gene may be created in this way, and the function of the gene studied in specific cells and/or at specific developmental stages.

Recombination between two site-specific recombinase recognition sequences in inverted repeat orientation may cause inversion of an intervening sequence or gene. Inversion may cause activation or inactivation of a gene. If gene activity is detectable (e.g., selectable marker, histochemical marker, reporter gene), cell lineages may be traced by identifying recombination events that mark a cell and its descendants through detection of gene activation or inactivation. Cell lineages may be traced independent of gene activity, by monitoring differences in the integration site of the site-specific recombinase recognition sequence.

Recombination between a site-specific recombinase recognition sequence integrated on a chromosome and a site-specific recombinase recognition sequence integrated on extrachromosomal genetic material may cause insertion of the genetic material into the chromosome. An insertion created in this manner would provide means for creating transgenic non-human animals with site-specific integration of a single copy of the transgene at a site in the genome specified by the chromosomal site-specific recombinase recognition sequence.

Preferably, the intervening sequence or genetic material contains a gene such as, for example, a developmental gene, essential gene, cytokine gene, neurotransmitter gene, neurotransmitter receptor gene, oncogene, tumor suppressor gene, selectable marker, or histochemical marker, or portion thereof. Recombination may cause activation or inactivation of a gene by juxtaposition of regulatory regions to the gene or separation of regulatory regions from the gene, respectively.

5.8. Exon Trapping

The piggyBac-like transposon systems of the invention may also be used in exon-trap cloning, or promoter trap procedures to detect differential gene expression in varieties of tissues. See, e.g., D. Auch & Reth, et al., “Exon Trap Cloning: Using PCR to Rapidly Detect and Clone Exons from Genomic DNA Fragments”, Nucleic Acids Research, Vol. 18, No. 22, p. 6743; Buckler, et al., 1996, Proc. Nat'l Acad. Sci. USA 88:4005-4009 (1991); Henske, et al., Am. J. Hum. Genet. 59:400-406. In such embodiments, the piggyBac-like transposon preferably comprises a detectable marker gene, such as GFP or an affinity tag, flanked by exon splicing donor and acceptor sites. The protein encoded by the marker gene is thus translated within the protein encoded by the genetic locus in which the piggyBac-like transposon is inserted, allowing for the detection of the protein encoded by the genetic locus.

5.9. Polypeptide Synthesis Applications

The methods described herein generating transgenic and mosaic animals and recombinant cells find use in the synthesis of polypeptides, e.g., proteins of interest.

In such applications, a transgenic or mosaic animal is generated, the genome of some or all its cells comprising a piggyBac-like transposon comprising an insert encoding the polypeptide of interest in combination with requisite and/or desired expression regulatory sequences, e.g., promoters, etc., (i.e., an expression module), to serve as an expression host for expression of the polypeptide. Similarly, a vertebrate cell in culture comprising such a piggyBac-like transposon can be used in these methods. The transgenic or mosaic animal or recombinant cell is then subjected to conditions sufficient for expression of the polypeptide encoded by the insert harbored by the piggyBac-like transposon. The expressed protein is then harvested, and purified where desired, using any convenient protocol.

In the context of a transgenic or mosaic animal, the methods of the invention provide a means for expressing a protein of interest in the animal or producing a cell line capable of high expression levels of a protein of interest. Thus, the animals and cells produced by the inventions are useful as “bioreactors” for the production of proteins of interest. The protein of interest can be endogenous or exogenous to the cells or animals.

Additionally, the methods described herein are useful improving traits of livestock.

5.10. Therapeutic Applications

The methods of the invention are useful in therapeutic applications, in which the piggyBac-like transposon systems are employed to stably integrate a therapeutic nucleic acid, e.g., gene, into the genome of a target cell, i.e., gene therapy applications. The piggyBac-like transposon systems may be used to deliver a wide variety of therapeutic nucleic acids to a subject. Therapeutic nucleic acids of interest include genes or open reading frames that replace defective genes in the target host cell, such as those responsible for genetic defect based diseased conditions; those which have therapeutic utility in the treatment of cancer; and the like. Exemplary therapeutically beneficial coding sequences are disclosed in Section 5.13.

An important feature of the subject methods, as described supra, is that the subject methods may be used for in vivo gene therapy applications. By in vivo gene therapy applications is meant that the target cell or cells in which expression of the therapeutic gene is desired are not removed from the host prior to contact with the transposon system. In contrast, vectors that include the transposon system are administered directly to the multicellular organism and are taken up by the target cells, following which integration of the gene into the target cell genome occurs.

5.11. Promoters

In one embodiment of the invention, the nucleic acid inserted into a piggyBac-like transposon encodes an open reading frame (“ORF”) operably linked to an element that regulates the expression of the ORF. Additionally, regulatory elements are desirable for regulating expression of the piggyBac-like transposase, particularly in embodiments of the invention in which a nucleic acid encoding a transposase is introduced into the genome of an animal.

Preferably, the expression module within a piggyBac-like transposon includes transcription regulatory elements that provide for expression of an ORF harbored by the transposon. Examples of specific transcription regulatory elements include: SV40 elements, as described in Dijketha et al., EMBO J. (1985) 4:761; transcription regulatory elements derived from the LTR of the Rous sarcoma virus, as described in Gorman et al., Proc. Nat'l Acad. Sci USA (1982) 79:6777; transcription regulatory elements derived from the LTR of human cytomegalovirus (CMV), as described in Boshart et al., Cell (1985) 41:521; hsp70 promoters, (Levy-Holtzman, R. and I. Schechter (Biochim. Biophys. Acta (1995) 1263: 96-98) Presnail, J. K. and M. A. Hoy, (Exp. Appl. Acarol. (1994) 18: 301-308)) and the like.

In specific embodiments, the regulatory element is an inducible promoter. Inducible promoters are known to those familiar with the art and a variety exists that could be used to drive expression of the transposase gene. Inducible systems include, for example, the heat shock promoter system, the metallothionein system, the glucocorticoid system, tissue specific promoters, etc. Promoters regulated by heat shock, such as the promoter normally associated with the gene encoding the 70-kDa heat shock protein, can increase expression several-fold after exposure to elevated temperatures. The glucocorticoid system also functions well in triggering the expression of genes. The system consists of a gene encoding glucocorticoid receptor protein (GR) which in the presence of a steroid hormone (i.e., glucocorticoid or one of its synthetic equivalents such as dexamethasone) forms a complex with the hormone. This complex then binds to a short nucleotide sequence (26 bp) named the glucocorticoid response element (GRE), and this binding activates the expression of linked genes. Thus inducible promoters can be used as an environmentally inducible promoter for controlling the expression of the introduced gene. Other means besides inducible promoters for controlling the functional activity of a gene product are known to those familiar with the art.

In certain embodiments, the piggyBac-like transposase is expressed under the control of a germline specific promoter. In certain embodiments, the germline specific promoter is a male-specific promoter (e.g., Protamine 1 (Prm) promoter, as described herein). In other embodiments, the germline specific promoter is a female-specific promoter (e.g., a ZP3 promoter, such as a murine ZP3 (mZP3) promoter (Lira et al., 1990, Proc. Nat'l. Acad. Sci. U.S.A. 87(18):7215-9).

For using livestock animals as bioreactors, protein can be produced in quantity in milk, urine, blood or eggs. Promoters are known that promote expression in milk, urine, blood or eggs and these include, but are not limited to, casein promoter, the mouse urinary protein promoter, (—globin promoter and the ovalbumin promoter respectively.

5.12. PiggyBac-Like Transposon Mutagenesis and Gene Discovery

Transposon tagging is a technique by which transgenic DNA is delivered to cells so that it will integrate into genes, thereby inactivating them by insertional mutagenesis. In the process, the inactivated genes are tagged by the transposable element which then can be used to recover the mutated allele. Insertion of a transposable element may disrupt the function of a gene which can lead to a characteristic phenotype.

Due to their inherent ability to move from one chromosomal location to another within and between genomes, transposable elements have revolutionized genetic manipulation of certain organisms including bacteria (Gonzales et al., 1996 Vet. Microbiol. 48, 283-291; Lee and Henk, 1996. Vet. Microbiol. 50, 143-148), Drosophila (Ballinger and Benzer, 1989 Proc. Natl. Acad. Sci. USA 86, 9402-9406; Bellen et al., 1989 Genes Dev. 3, 1288-1300; Spradling et al., 1995 Proc. Natl. Acad. Sci. USA 92, 10824-10830), C. elegans (Plasterk, 1995. Meth. Cell. Biol., Academic Press, Inc. pp. 59-80) and a variety of plant species (OpiggyBac-likeorne and Baker, Curr. Opin. Cell Biol, 7, 406-413 (1995)). Transposons have been harnessed as useful vectors for transposon-tagging, enhancer trapping and transgenesis. However, the majority, if not all, vertebrates lack such a powerful tool. For their simplicity and ability to function in diverse organisms, the piggyBac-like transposon systems of the invention are useful as an efficient vector for species in which DNA transposon technology is currently not available.

Transposon tagging is a technique in which transposons are mobilized to “hop” into genes, thereby inactivating them by insertional mutagenesis. These methods are discussed by Evans et al., TIG 1997 13:370-374. In the process, the inactivated genes are “tagged” by the transposable element which then can be used to recover the mutated allele. Therefore, the present invention provides an efficient method for introducing a piggyBac-like transposon tag into the genome of a cell. Where the tag is inserted into a location in the cell that disrupts expression of a protein that is associated with a particular phenotype, expression of an altered phenotype in a cell containing the piggyBac-like transposon permits the association of a particular phenotype with a particular gene that has been disrupted by transposon. Here the piggyBac-like transposon functions as a tag. Primers designed for inverse PCR or to sequence the genomic DNA flanking the nucleic acid fragment of this invention can be used to obtain sequence information about the disrupted gene.

There are several ways of isolating the tagged gene. In all cases genomic DNA is isolated from cells from one or more tissues of the mutated animal by conventional techniques (which vary for different tissues and animals). The DNA is cleaved by a restriction endonuclease that may or may not cut in the transposon tag (more often than not it does cleave at a known site). The resulting fragments can then either be directly cloned into plasmids or phage vectors for identification using probes to the transposon DNA (see Kim et al., 1995 for references in Mobile Genetic Elements, IRL Press, D. L. Sheratt eds.). Alternatively, the DNA can be PCR amplified in any of many ways. The LM-PCR procedure of Izsvak and Ivics (1993, Biotechniques. 15(5):814-8) can be used. The LM-PCR procedure can be performed as modified by Devon et al. (1995, Nucleic Acids Res. 23(9):1644-5) and identified by its hybridization to the transposon probe. An alternative method is inverse-PCR (e.g., Allende et al., 1996, Genes Dev., 10:3141-3155). Regardless of method for cloning, the identified clone is then sequenced. The sequences that flank the transposon (or other inserted DNA) can be identified by their non-identity to the insertional element. The sequences can be combined and then used to search the nucleic acid databases for either homology with other previously characterized gene(s), or partial homology to a gene or sequence motif that encodes some function. In some cases the gene has no homology to any known protein. It becomes a new sequence to which others will be compared. The encoded protein will be the center of further investigation of its role in causing the phenotype that induced its recovery.

Thus, piggyBac-like transposons can be employed to mutagenize vertebrate genomes, allowing the generation of loss-of-function mutants and screening the mutants for phenotypes of interest. Typically, the piggyBac-like transposons are used which contain one or more elements that allow detection of animals containing the transposon. Most often, marker genes are used that affect a visible trait, such as coat or eye color. However, any gene can be used as a marker that causes a reliable and easily scored phenotypic change in transgenic animals.

A gene into which a piggyBac-like transposon is inserted can be identified by digesting the DNA of the cell into which the transposon is inserted with a restriction endonuclease capable of cleaving the piggyBac-like transposon sequence; identifying the inverted repeat sequences of the transposon; sequencing the nucleic acid close to the inverted repeat sequences to obtain DNA sequence from an open reading frame; and comparing the DNA sequence with sequence information in a computer database. In one embodiment, the restriction endonuclease recognizes a 4-base recognition sequence. In another embodiment, the digesting step further comprises cloning the digested fragments or PCR amplifying the digested fragments. In one embodiment, the gene is identified by inverse PCR.

Thus, the piggyBac-like transposon systems of the invention can also be used for gene discovery. In one example, the piggyBac-like in combination with the piggyBac-like transposase protein or nucleic acid encoding the piggyBac-like transposase is introduced into a cell. The piggyBac-like transposon preferably comprises an insert that includes a marker protein, such as GFP and a restriction endonuclease recognition site, preferably a 6-base recognition sequence. Following integration, the cell DNA is isolated and digested with the restriction endonuclease. Where a restriction endonuclease is used that employs a 4-base recognition sequence, the cell DNA is cut into about 256-bp fragments on average. These fragments can be either cloned or linkers can be added to the ends of the digested fragments to provide complementary sequence for PCR primers. Where linkers are added, PCR reactions are used to amplify fragments using primers from the linkers and primers binding to the direct repeats of the inverted repeats in the nucleic acid fragment. The amplified fragments are then sequenced and the DNA flanking the direct repeats is used to search computer databases such as GenBank.

5.12.1. Phenotypic Reversion for Mutation Verification

The piggyBac-like transposons employed in the methods of the invention excise precisely upon transposition in vivo, without leaving behind any of the transposon sequence upon excision. This feature of the piggyBac-like transposon system can be taken advantage of to confirm that a phenotype observed in a non-human vertebrate directly results from the insertion of a piggyBac-like transposon into the genome.

5.13. Gene Therapy

Gene transfer vectors for gene therapy can be broadly classified as viral vectors or non-viral vectors. The use of the piggyBac-like transposon system is a refinement of non-viral DNA-mediated gene transfer. Up to the present time, viral vectors have been found to be more efficient at introducing and expressing genes in cells. There are several reasons why non-viral gene transfer is superior to virus-mediated gene transfer for the development of new gene therapies. For example, adapting viruses as agents for gene therapy restricts genetic design to the constraints of that virus genome in terms of size, structure and regulation of expression. Non-viral vectors are generated largely from synthetic starting materials and are therefore more easily manufactured than viral vectors. Non-viral reagents are less likely to be immunogenic than viral agents making repeat administration possible. Non-viral vectors are more stable than viral vectors and therefore better suited for pharmaceutical formulation and application than are viral vectors.

Current non-viral gene transfer systems are not equipped to promote integration of nucleic acid into the DNA of a cell, including host chromosomes. As a result, stable gene transfer frequencies using non-viral systems have been very low; 0.1% at best in tissue culture cells and much less in primary cells and tissues. The present system is a non-viral gene transfer system that facilitates integration and markedly improves the frequency of stable gene transfer.

In the gene transfer system of this invention the piggyBac-like transposase can be introduced into the cell as a protein or as nucleic acid encoding the protein. In one embodiment the nucleic acid encoding the protein is RNA and in another, the nucleic acid is DNA. Further, nucleic acid encoding the piggyBac-like transposase can be incorporated into a cell through a viral vector, cationic lipid, or other standard transfection mechanisms including electroporation or particle bombardment used for eukaryotic cells. Following introduction of nucleic acid encoding the piggyBac-like transposon, the piggyBac-like transposase can be introduced into the same cell.

Similarly, the piggyBac-like transposase can be introduced into the cell as a linear fragment or as a circularized fragment, preferably as a plasmid or as recombinant viral DNA. Preferably the nucleic acid sequence comprises at least a portion of an open reading frame to produce an amino-acid containing product. In a preferred embodiment the piggyBac-like transposon comprises an insert that encodes at least one protein, for example a selectable marker, a reporter, a therapeutic protein or a protein of value in the livestock industry, and includes at least one promoter selected to direct expression of the open reading frame or coding region inserted into the piggyBac-like transposon. A more extensive description of the suitable coding regions contained in the piggyBac-like transposons of the invention are provided in Section 5.14, infra.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215). Methods commonly known in the art of recombinant DNA technology which can be used are described in Ausubel et al. (eds.), 1993, Current Protocols in Molecular Biology, John Wiley & Sons, New York; and Kriegler, 1990, Gene Transfer and Expression, A Laboratory Manual, Stockton Press, New York. Any such methods can be use to deliver a piggyBac-like nucleic acid of the invention.

Delivery of the piggyBac-like nucleic acid, e.g., a nucleic acid comprising a piggyBac-like transposon and/or nucleotide sequence encoding a piggyBac-like transposase, optionally operably linked to a promoter, into a patient may be either direct, in which case the patient is directly exposed to the nucleic acid or nucleic acid-carrying vector, or indirect, in which case, cells are first transformed with the piggyBac-like nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., Wu and Wu, 1987, J. Biol. Chem. 262:4429-4432) (which can be used to target cell types specifically expressing the receptors), etc. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, a viral vector that contains the piggyBac-like nucleic acid is used. For example, a retroviral vector can be used (see Miller et al., 1993, Meth. Enzymol. 217:581-599). These retroviral vectors have been modified to delete retroviral sequences that are not necessary for packaging of the viral genome and integration into host cell DNA. The piggyBac-like nucleic acid to be used in gene therapy is cloned into the vector, which facilitates delivery of the gene into a patient. More detail about retroviral vectors can be found in Boesen et al., 1994, Biotherapy 6:291-302. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; and Mastrangeli et al., 1993, J. Clin. Invest. 91:225-234.

Adeno-associated virus (AAV) has also been proposed for use in gene therapy (Walsh at al., 1993, Proc. Soc. Exp. Biol. Med. 204:289-300.

Another approach to gene therapy involves transferring a piggyBac-like nucleic acid to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the piggyBac-like nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. In a preferred embodiment, epithelial cells are injected, e.g., subcutaneously. In another embodiment, recombinant skin cells may be applied as a skin graft onto the patient. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a piggyBac-like nucleic acid can be introduced for purposes of gene therapy encompass any desired, available cell types, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

5.14. Proteins Encoded by PiggyBac-Like Transposons

As discussed herein, the piggyBac-like transposons of the invention are useful for delivering a variety of nucleic acids that are harbored by the nucleic acids to a subject. Additionally, in certain applications such as enhancer trapping, the transposons may usefully harbor markers genes. In yet other aspects, the piggyBac-like transposons can harbor a nucleotide sequence that modifies a trait in the genome of the target cell or organism, a selectable marker, etc. Examples of such nucleic acids harbored by the piggyBac-like transpsons of the invention are provided below.

Specific therapeutic genes for use in the treatment or prevention of genetic defect based disease conditions include genes encoding the following products: factor IX, β-globin, low-density protein receptor, adenosine deaminase, purine nucleoside phosphorylase, sphingomyelinase, glucocerebrosidase, cystic fibrosis transmembrane regulator, α-antitrypsin, CD18, ornithine transcarbamylase, arginosuccinate synthetase, phenylalanine hydroxylase, branched-chain α-ketoacid dehydrogenase, fumarylacetoacetate hydrolase, glucose 6-phosphatase, α-L-fucosidase, β-glucuronidase, α-L-iduronidase, galactose 1-phosphate uridyltransferase, insulin, human growth hormone, erythropoietin, clotting factor VII, bovine growth hormone, platelet derived growth factor, clotting factor VIII, thrombopoietin, interleukin-1, interluekin-2, interleukin-1 RA, superoxide dismutase, catalase, fibroblast growth factor, neurite growth factor, granulocyte colony stimulating factor, L-asparaginase, uricase, chymotrypsin, carboxypeptidase, sucrase, calcitonin, Ob gene product, glucagon, interferon, transforming growth factor, ciliary neurite transforming factor, insulin-like growth factor-1, granulocyte macrophage colony stimulating factor, brain-derived neurite factor, insulintropin, tissue plasminogen activator, urokinase, streptokinase, adenosine deamidase, calcitonin, arginase, phenylalanine ammonia lyase, .gamma.-interferon, pepsin, trypsin, elastase, lactase, intrinsic factor, cholecystokinin, and insulinotrophic hormone, and the like.

Cancer therapeutic genes that may be delivered via the subject methods include: genes that enhance the antitumor activity of lymphocytes, genes whose expression product enhances the immunogenicity of tumor cells, tumor suppressor genes, toxin genes, suicide genes, multiple-drug resistance genes, antisense sequences, and the like.

Marker gene sequences harbored by the piggyBac-like transposons of the invention can be an enzyme, a protein or peptide comprising an epitope, a receptor, a transporter, tRNA, rRNA, or a bioluminescent, chemiluminescent or fluorescent molecule. In specific embodiments, the marker is green fluorescent protein (GFP) or a mutant thereof, such as a mutant GFP having an altered fluorescence wavelength, increased fluorescence, or both. In certain specific embodiment, the mutant GFP is blue GFP. In other modes of the embodiment, the fluorescent molecule is red fluorescent protein (see Section 6) or yellow fluorescent protein. In yet other embodiments, the marker is chloramphenicol acetyltransferase (CAT), β-galactosidase (lacZ), and luciferase (LUC).

In livestock uses, the piggyBac-like transposons can harbor sequences for growth hormones, such as insulin-like growth factors (IGFs), for example to promote growth in a transgenic animal. In other livestock uses, the transgene harbored by the piggyBac-like transpson can provide greater resistance to disease.

A number of marker genes can be inserted into the piggyBac-like transposons into the invention, including but not limited to the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk-, hgprt- or aprt-cells, respectively. Also, antimetabolite resistance can be used as the basis of selection for dhfr, which confers resistance to methotrexate (Wigler et al., 1980, Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); hygro, which confers resistance to hygromycin (Santerre et al., 1984, Gene 30:147); trpB, which allows cells to utilize indole in place of tryptophan; hisD, which allows cells to utilize histinol in place of histidine (Hartman & Mulligan, 1988, Proc. Natl. Acad. Sci. USA 85:8047); and ODC (ornithine decarboxylase) which confers resistance to the ornithine decarboxylase inhibitor, 2-(difluoromethyl)-DL-ornithine, DFMO (McConlogue, L., 1987, In: Current Communications in Molecular Biology, Cold Spring Harbor Laboratory, Ed.).

5.15. Non-Coding Sequences Harbored by PiggyBac-Like Transposons

In addition, or as an alternative to, an ORF, the piggyBac-like transposon of the present invention may also include at least one sequence that is recognized by a protein that binds to and/or modifies nucleic acids. In specific embodiments, the protein is a DNA-binding protein, a DNA-modifying protein, an RNA-binding protein, or an RNA-modifying protein.

In certain specific embodiments, the sequence is one that is recognized by a restriction endonuclease, i.e., a restriction site. A variety of restriction sites are known in the art and may be included, for example sites recognized by the following restriction enzymes: HindIII, PstI, SalI, AccI, HincII, XbaI, BamHI, SmaI, XmaI, KpnI, SacI, EcoRI, and the like.

In other specific embodiments, the sequence is a target site for a site-specific recombinase, such as FLP recombinase (i.e., the sequence is a FRT) or the CRE recombinase (i.e., the sequence is a loxP). Such embodiments are useful to generate mosaic animals, as described in Section 5.7).

5.16. Veterinary and Livestock Uses of the Invention

The present methods and compositions can be utilized in a non-human animal for a veterinary use for treating or preventing a disease or disorder or for improving the quality of livestock.

In a specific embodiment, the non-human animal is a household pet. In another specific embodiment, the non-human animal is a livestock animal. In a preferred embodiment, the non-human animal is a mammal, most preferably a cow, horse, sheep, pig, cat, dog, mouse, rat, rabbit, hamster, mink, or guinea pig. In another preferred embodiment, the non-human animal is a fowl species, most preferably a chicken, turkey, duck, goose, or quail.

### 6. EXAMPLES

### 6.1. Introduction

Transposable elements have been routinely used as tools for genetic manipulations in lower organisms, including the generation of transgenic animals and insertional mutagenesis. In contrast, the usage of transposons in mice and other vertebrate systems is still limited due to the lack of an efficient transposon system. We have tested the ability of piggyBac, a DNA transposon from the cabbage looper moth Trichoplusia ni, to transpose in mammalian systems, and have found that piggyBac elements carrying multiple genes can efficiently transpose in human and mouse cell lines and also in mice. The data presented herein indicate that during germline transposition the piggyBac elements excise precisely from original insertion sites and transpose into the mouse genome at diverse sites, preferably transcription units, and permitted the expression of the marker genes carrying by the transposon. These data provide a critical step towards a highly efficient transposon system for a variety of genetic manipulations including transgenesis and insertional mutagenesis in mice and other vertebrates.

### 6.2. Materials and Methods

6.2.1. Plasmid Construction

PB[SV40-neo]: The BamHI-KpnI fragment of pSLfa1180fa (Horn and Wimmer, 2000, Dev Genes Evol 210, 630-637) was replaced by the BamHI-KpnI fragment from pCLXSN (IMGENEX). The neomycin cassette was then cut out with AscI and inserted into the AscI site of pBac{3×P3-EGFPafm} (Horn and Wimmer, 2000, Dev. Genes Evol. 210:630-637).

CMV-PBase: The coding sequence of the piggyBac transposase was PCR amplified from phsp-Bac (Handler and Harrell, 2001, Insect Biochem Mol Biol 31:199-205) with primers BacEN-F (5′-GCCACCATGGGATGTTCTTTAG-3′) (SEQ ID NO:1) and BacEN-B (5% GTACTCAGAAACAACTTTGGC-3′) (SEQ ID NO:2), and cloned into the SpeI and SphI sites of pSLfa1180fa to generate pSL-BacEN. A HindIII-EcoRI fragment containing the transposase gene was isolated from pSL-BacEN and inserted into pcDNA4/HisA (Invitrogen) to generate the final construct.

PB/PGK-neol: The PGK-neo gene from pPNT (Tybulewicz et al., 1991, Cell 65:1153-1163) was cloned into the BglII site of pBac-AB, a modified piggyBac construct to generate PB[PGK-neo].

PB[Act-RFP]: The 0.7 kb EcoRI fragment of pCX-EGFP (Okabe et al., 1997, FEBS Lett 407:313-319) was replaced by the coding sequence of mRFP (Campbell et al., 2002, Proc. Nat'l. Acad. Sci. USA 99:7877-7882) to make pCX-RFP. The SaII-BamHI fragment of the pCX-RFP including the intact RFP expression cassette was further cloned into the BglII site of pBac-AB to generate PB[Act-RFP]. Polylinkers were added to generate the universal PB vector PB[Act-RFP]DS, which has multiple unique cloning sites.

Prm1-PBase: The Pmr-1 promoter and the BamHI-SalI fragment from pPrm1-SB10 (Fischer et al., 2001, Proc. Nat'l. Acad. Sci. USA 98:6759-6764) were cloned into the HindIII site and the BamHI-XhoI site of pSL-BacEN, receptively, to generate this testis specific transposase helper plasmid.

Act-PBase: Using a NheI-NotI linker, the EcbRI fragment of pCX-EGFP was replaced by the SpeI-EagI transposase fragment of pSL-BacEN to generate this ubiquitously expressed transposase helper plasmid.

PB[K14-Tvr]: The SmaI fragment of K14 promoter in pinK14-Albino (Saitou et al., 1995, Nature 374:159-162), a tyrosinase cDNA amplified from a skin sample of a 129Sv mouse by RT-PCR, and the SV40 polyA, were inserted into the BglII site of pBac-AB to generate PB[K14-Tyr].

PB[K14-Tvr, Act-RFP]: The SalI-BamHI fragment from pCX-RFP was cloned into the AscI site of PB[K14-Tyr] to generate this construct.

PB[Act-RFP, MCK-TSC1]: The SmaI fragment from PB[Act-RFP], that consists of the RFP expression cassette and the left terminus (piggyBacL) was used to replace the SalI-EcoRV fragment of pBluescript to generate pBS-BLRFP. The SmaI-EcoRV fragment of PB[Act-RFP]DS, that consists of the right terminus (PBR), was then cloned into the PmeI site of pBS-BLRFP to generatePB[Act-RFP], which serves as a universal piggyBac-based transgenic vector. The BssHII fragment of the MCK-TSC1 construct (Inoki et al., 2002, Nat. Cell Biol. 4:648-657) and a hGH polyA (Nguyen et al., 1998, Science 279:1725-1729) was cloned into the SwaI site of PB[Act-RFP]DS.

6.2.2. Cell Transfections

293 cells were cultured in DMEM (GIBCO/BRL) supplemented with 10% serum at 37° C. and 5% CO2. 1.5×10⁵ cells were seeded into each well of a 24-well-plate one day prior to transfection. For each well, 0.5 μg circular PB[SV40-neo] and 0.5 μg circular CMV-PBase in test group or 0.5 us circular pcDNA4/HisA in control group were transfected by LipofectAMINE 2000 according to the standard protocol (Invitrogen). One day after the transfection, the cells in each well were trypsinized and seeded onto one 10-cm plate in medium containing 500 mg/ml G-418 (GIBCO/BRL). Drug selection continued for two weeks.

The conditions for culture and electroporation of W4/129S6 mouse embryonic stem (ES) cells were described in the manufacturer recommended protocols (Taconic). Twenty-four micrograms of circular PB[PGK-neo] and 6 μg Act-PBase in the test group or 6 μg herring sperm DNA (Promega) in the control group were used for electroporation of ten million cells. Immediately after eletroporation, cells in each group were seeded onto three 10-cm plates containing mitomycin C treated mouse embryonic fibroblast feeder cells. Selection was initiated 48 hours after electroporation with medium containing 200 mg/ml G-418. Drug selection continued for two weeks.

At the end of drug selection, cells were fixed with PBS containing 4% paraformaldehyde for 10 minutes and then stained with 0.2% methylene blue for one hour. Clones were counted after extensive washing with deionized water.

6.2.3. PCR and Sequence Analysis

HaeIII or MspI digests of genomic DNA were self-ligated to serve as the template for inverse PCR. Primers used to recover the flanking sequence of the left side of the piggyBac transposon were LF1 (5′-CTT GAC CTT GCC ACA GAG GAC TAT TAG AGG-3′) (SEQ ID NO:3) and LR1 (5′ CAG TGA CAC TTA CCG CAT TGA CAA GCA CGC-3′) (SEQ ID NO:4). Primers used to recover the flanking sequence of the right side of piggyBac transposon were RF1 (5′-CCT CGA TAT ACA GAC CGA TAA AAC ACA TGC-3′) (SEQ ID NO:5) and RR1 (5′-AGT CAG TCA GAA ACA ACT TTG GCA CAT ATC-3′) (SEQ ID NO:6).

PCR detection of excision site was carried out with primer EL1 (5′-CCA TAT ACG CAT CGG GTT GA-3′) (SEQ ID NO:7) and primer ER1 (5′-TTA AAG TTT AGG TCG AGT AAA GCG C-3′) (SEQ ID NO:8).

PCR products were cloned into pGEM-T vector (Promega) for subsequent sequencing. Sequencing results were analyzed with NCBI BLAST searches (www.ncbi.nlm.nih.gov) and Ensembl human or mouse genome databases (www.ensembl.org).

To detect additional sequence preferences of PB insertion events, five base pairs upstream and downstream of the TTAA target site were analyzed for 100 piggyBac insertions in mice. At the same time, 100 randomly selected TTAA sites were analyzed as the control. One-sided probabilities were calculated between two proportions with STATISTICA 6.0.

6.2.4. Generation of Transgenic Mice

Circular piggyBac donor constructs were mixed with a helper plasmid at a ratio of 2:1. Mixed DNA samples (2 ng/μl) were microinjected into the fertilized FVB/Nj oocytes as described (Nagy at al., 2003, Manipulating the mouse embryo: a laboratory manual, 3rd edition (Cold Spring Harbor Laboratory Press)).

6.2.5. Southern Blot

Genomic DNA was isolated from tail samples, digested with EcoRV and BglII, and then fractionated in 0.7% agarose gels prior to Southern analysis. The probe was a 499 by fragment of SadII digest of PB[Act-RFP].

### 6.3. Results

6.3.1. Transposition Activity of PiggyBac in Cultured Mammalian Cells

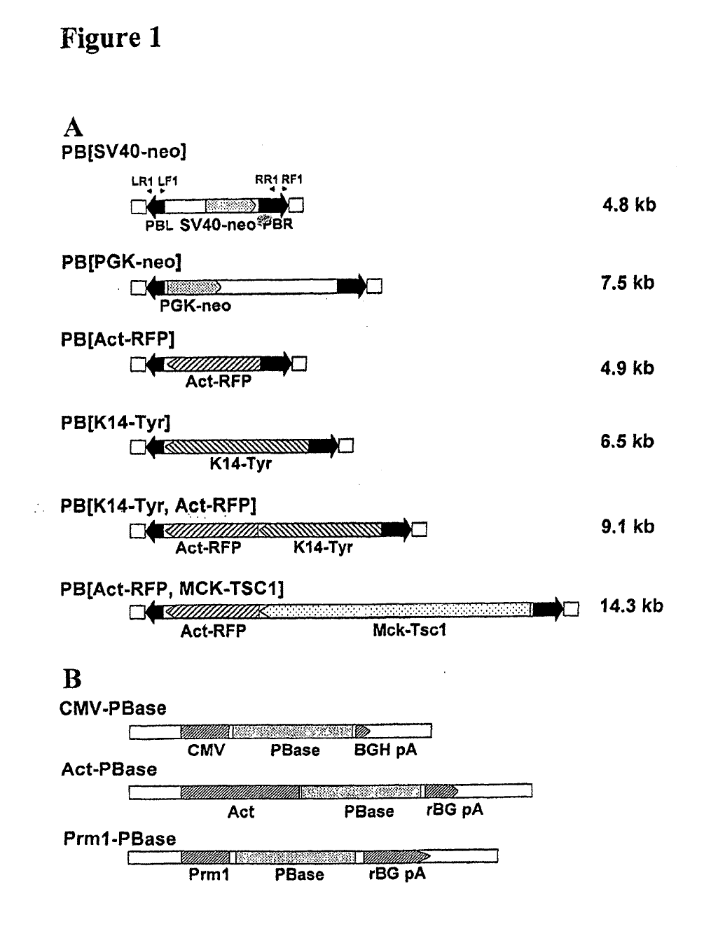

A binary co-transfection assay system consisting of both a donor and a helper plasmid was designed to detect piggyBac mediated chromosomal integration events in tissue culture cells. The donor plasmid contained the piggyBac elements in which the piggyBac transposase (PBase) was replaced by a drug selection marker (FIG. 1A). The helper plasmid carried the transposase fragment but lacked the terminal sequences required for transposition (FIG. 1B). In the absence of the helper plasmid, the donor plasmid may randomly integrate into the genome, but these random integration events can be minimized if the plasmid is kept in circular form. Thus, an increase of drug resistant clones in the presence of helper plasmid would indicate transposition events.

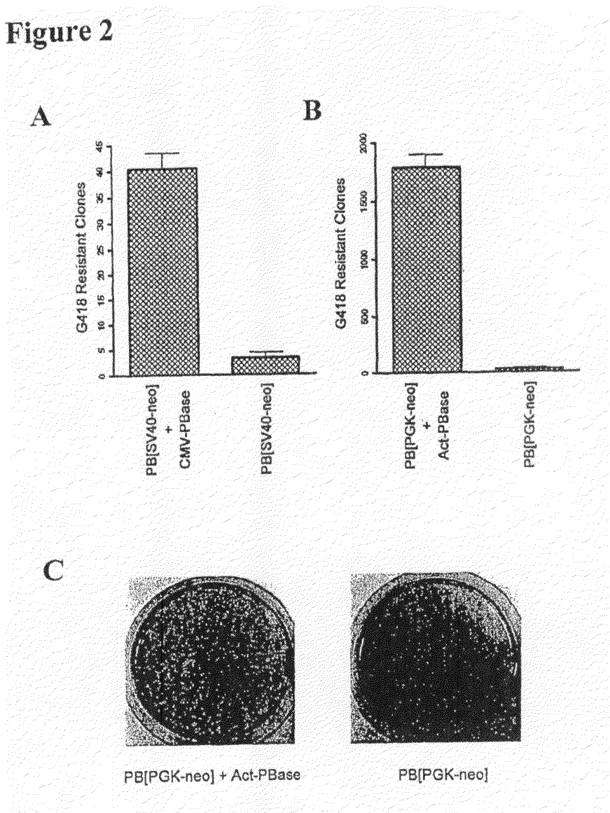

We first examined piggyBac transposition in human 293 cells. Co-transfection of the donor PB[SV40-neo] element carrying a SV40 promoter driven neomycin resistance (neo) gene and the helper CMV-PBase carrying a ubiquitously expressed transposase (FIG. 1) produced neomycin-resistant clones 10-fold higher than transfection with donor plasmid alone (FIG. 2A). To test whether the elevated integration of donor was due to transposition, inverse PCR was performed to recover sequence adjacent to the piggyBac right inverted terminal repeat (PBR) site of integrated PB[SV40-neo] (FIG. 1A). PCR products from a true transposition event should result in genomic sequence outside the PBR rather than plasmid sequence. Eighteen independent genomic sequences were recovered from five drug resistant clones. All of these sequences contained the signature TTAA sequence at the integration site (Table 2).

TABLE 2
PB transposition in human 293 cells

| Insertion No. | Insertion Site | Chromosome | Gene Name/Ensemble ID | Insertion Position |
|---|---|---|---|---|
| PBE 1T-3 | TTAAAGAAACACAG | 4 | NM_003603 | intron |
| | (SEQ ID NO: 9) | | | |
| PBE 1T-6 | TTAATAAAGGGGTT | repeats (MER7A) | | |
| | (SEQ ID NO: 10) | | | |
| PBE 1T-5 | TTAAAGCTCCAAA | repeats | | |
| | (SEQ ID NO: 11) | | | |
| PBE 1T-7 | TTAAAAAAATTTAT | 12 | Q9Y219 | intron |
| | (SEQ ID NO: 12) | | | |
| PBE 1T-11 | TTAAAGAATCATGG | 6 | | NA |
| | (SEQ ID NO: 13) | | | |
| PBE 1T-13 | TTAATACAACTTGC | 7 | | intergenic |
| | (SEQ ID NO: 14) | | | |
| PBE 1T-15 | TTAAAACGGAAGTT | 2 | ERBB4 | intron |
| | (SEQ ID NO: 15) | | | |
| PBE 1T-29 | TTAAGTAATAATAA | Repeats (Alu) | | |
| | (SEQ ID NO: 16) | | | |
| PBE 1T-36 | TTAAAAGCTAAGCC | 3 | | intergenic |
| | (SEQ ID NO: 17) | | | |
| PBE 2T-6 | TTAATTAATCTGGG | 14 | | NA |
| | (SEQ ID NO: 18) | | | |
| PBE 2T-10 | TTAAGCCTATACCC | 3 | | NA |
| | (SEQ ID NO: 19) | | | |
| PBE 3T-2 | TTAAAGTAAGAAAT | 19 | XM-371190 | intron |
| | (SEQ ID NO: 20) | | | |
| PBE 3T-7 | TTAAAGGAATACCA | 18 | | intergenic |
| | (SEQ ID NO: 21) | | | |
| PBE 3T-11 | TTAACCCTCCTAAC | 17 | ENSESTT00000051373 | intron |
| | (SEQ ID NO: 22) | | | |

| | | |
|---|---|---|
| PBE 6T-2 | TTAAAGATCAAAGT | repeats (MER61E-int) |
| | (SEQ ID NO: 23) | |

| | | | | |
|---|---|---|---|---|
| PBE 6T-4 | TTAATAATTTGTCC | 22 | PLA2G6 | intron |
| | (SEQ ID NO: 24) | | | |
| PBE 7T-1 | TTAAAGAATGGTTA | 22 | QBTC68 | intron |
| | (SEQ ID NO: 25) | | | |
| PBE 7T-7 | TTAAAAGACCTTTA | repeats (VERVH) | | |
| | (SEQ ID NO: 26) | | | |

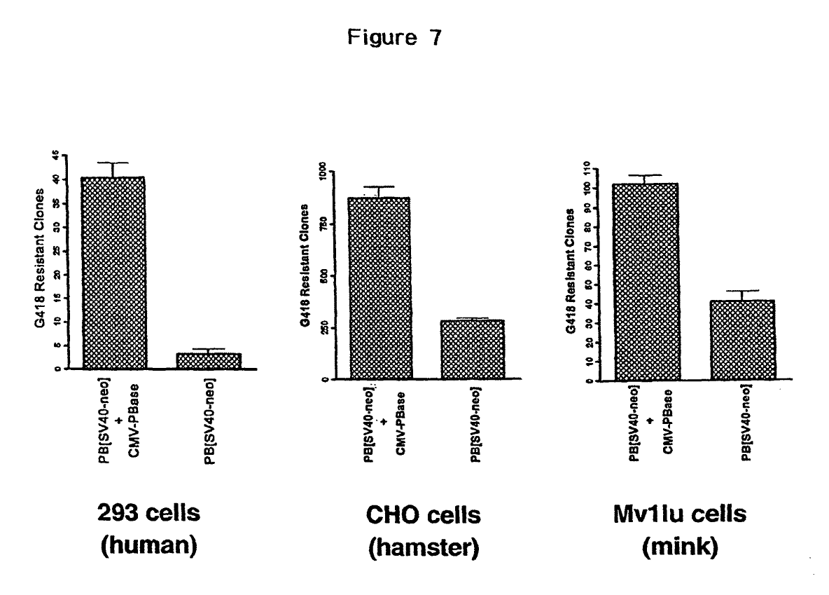

TTAA duplication was confirmed by sequencing several junction fragments at the other end of the transposon (data not shown). In contrast, inverse PCR analysis of neomycin resistant clones stably transfected with PB[SV40-neo] alone only detected junction plasmid sequences, which is consistent with random insertion events (data not shown). This experiment demonstrated that piggyBac transposition occurred in human cells with the same site-preference as in insect cells. Similar results were obtained when the co-transfection procedure was carried out in Chinese Hamster Ovary (CHO) cells and Mv1Lu cells (of mink origin) (see FIG. 7).

We next tested the ability of piggyBac to transpose in mouse W4/129S6 embryonic stem (ES) cells. In this test, the donor plasmid PB[PGK-neo] element carried a PGK promoter driven neo gene and the helper plasmid Act-PBase provided piggyBac transposase under the control of a hybrid actin promoter (FIG. 1B). In three repeated transfection experiments, PB[PGK-neo] and Act-PBase co-transfection produced drug resistant clones on average 50-fold higher than PB[PGK-neo] transfection alone (FIGS. 2B and 2C). Inverse PCR analysis confirmed that the enhanced clone production was due to transposition (Table 3).

TABLE 3
PB transposition in mouse W4/129S6 embryonic stem cells.

| | | | | |
|---|---|---|---|---|
| Insertion No. | Insertion Site | Chromosome | Gene Name/Ensemble ID | Insertion Position |

| | | | | |
|---|---|---|---|---|
| PBES2T1 | TTAAGTTGTACCAA | 2 | B230339M05Rik | intron |
| | (SEQ ID NO: 27) | | | |
| PBES2T3 | TTAAAGGAGAGACT | 1 | GENSCAN00000093186 | intron |
| | (SEQ ID NO: 28) | | | |
| PBES2T4 | TTAACTGCCCAGTG | repeats (LTRs) | | |
| | (SEQ ID NO: 29) | | | |
| PBES4T58 | TTAACAAAACAAAA | 6 | 4833415F11Rik | exon |
| | (SEQ ID NO: 30) | | | |
| PBES4T59 | TTAATCAACAAATA | 5 | | intergenic |
| | (SEQ ID NO: 31) | | | |
| PBES4T63 | TTAAAGAGTCCCCT | 2 | NoI5a | intron |
| | (SEQ ID NO: 32) | | | |
| PBES9T27 | TTAACAACAGATAA | 5 | | intergenic |
| | (SEQ ID NO: 33) | | | |

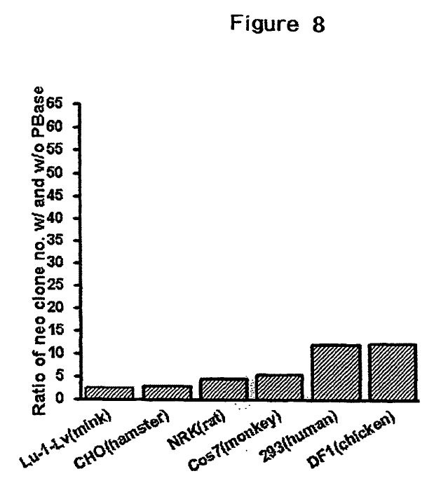

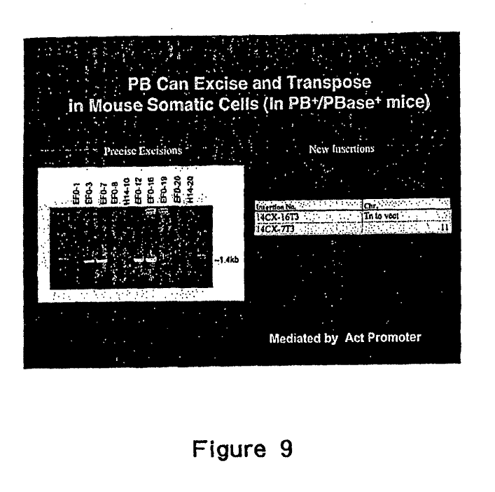

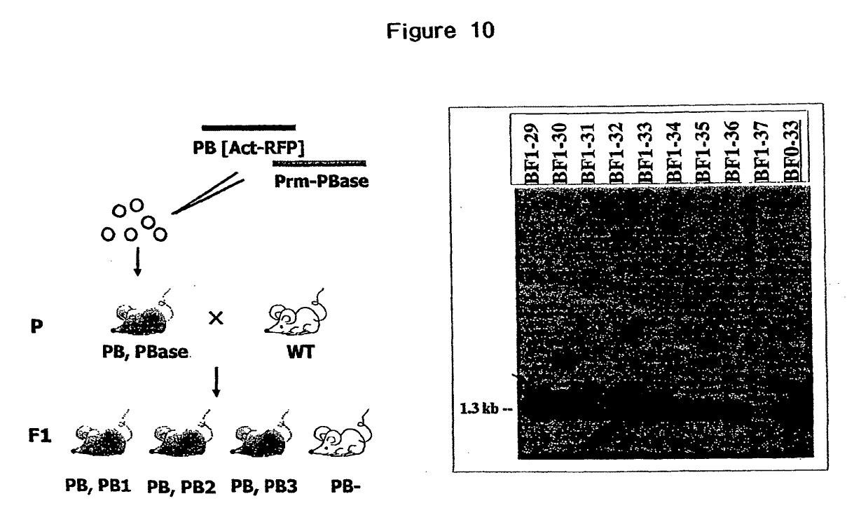

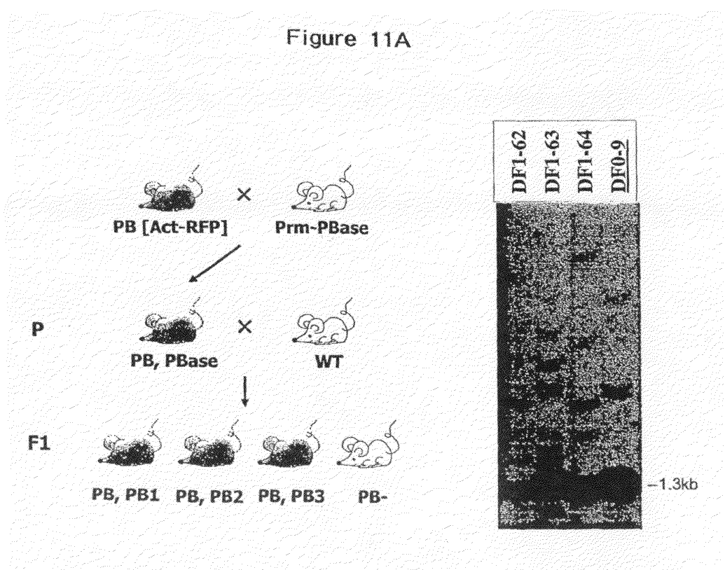

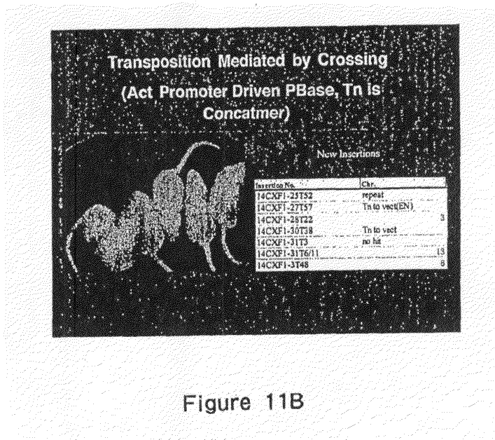

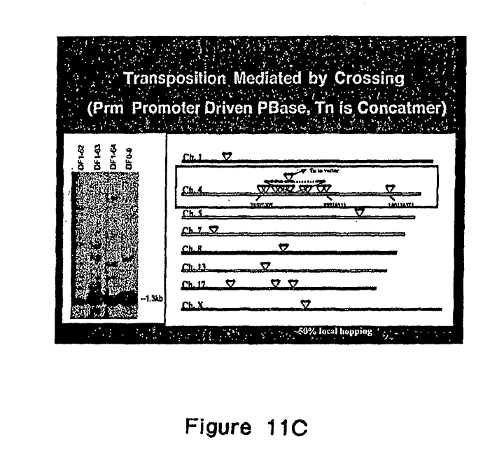

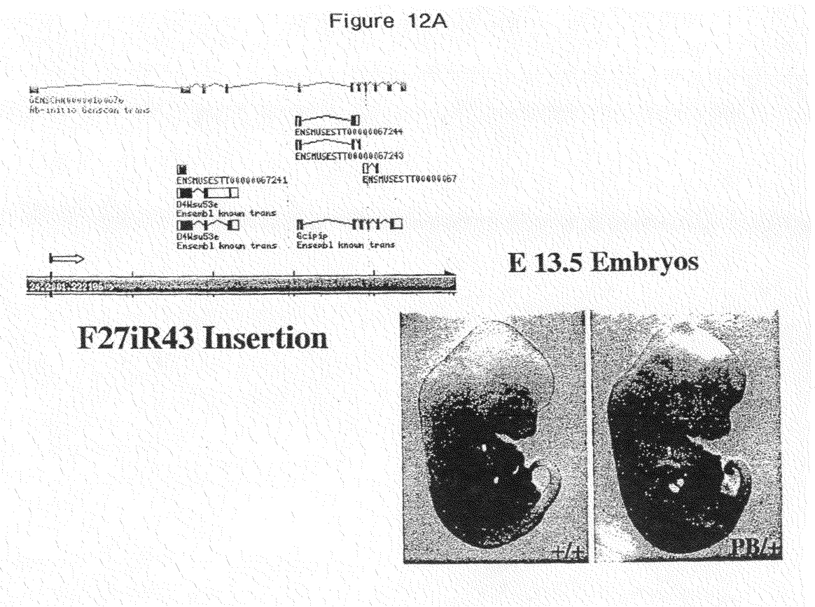

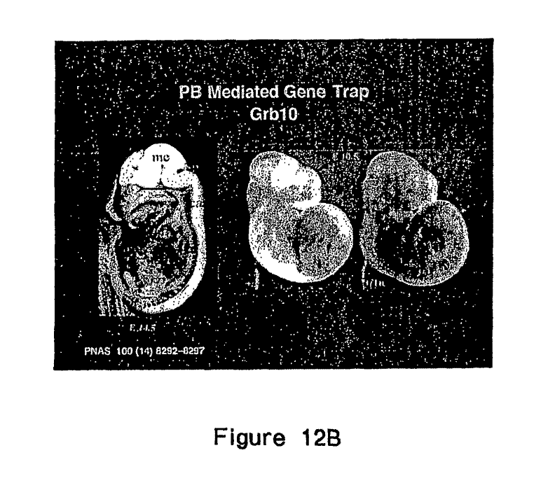

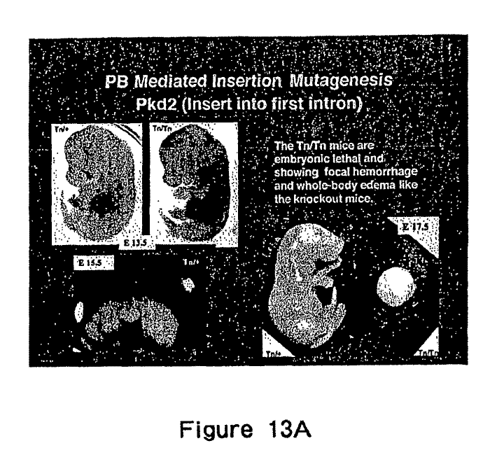

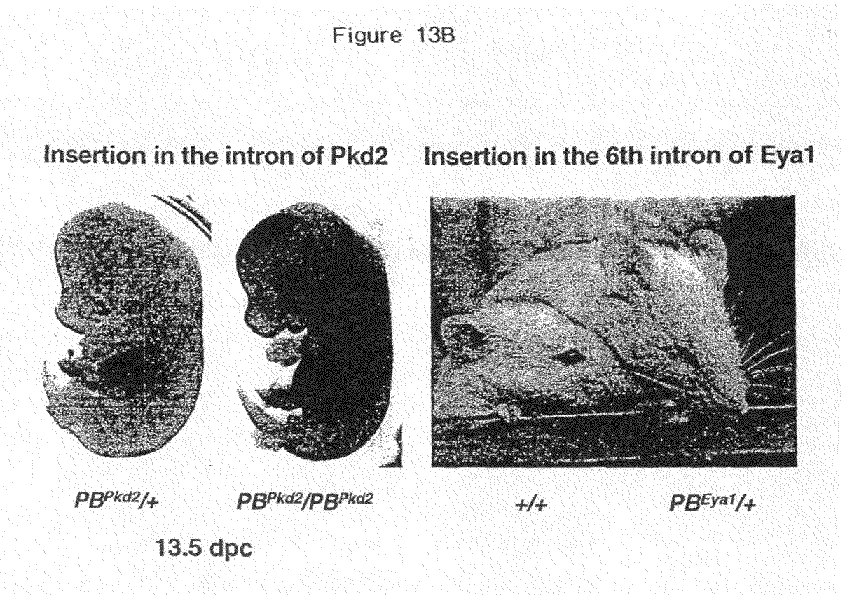

Similar transposition results were obtained when the co-transfection procedure was carried out in a variety of cell lines of different origins, including mink, hamster, rat, monkey, human and chicken (see FIG. 8).

6.12. PiggyBac Transposes Efficiently in the Mouse Germline

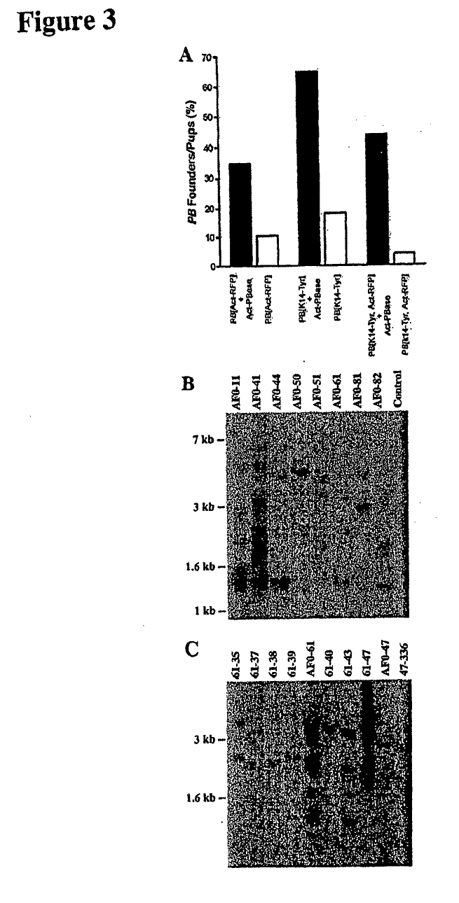

Efficient transposition in mouse ES cells encouraged us to test the feasibility of piggyBac transposition in the mouse germline. Pronuclei co-injection of transposon donor and transposase helper plasmids was performed to generate transgenic mice. To facilitate the analysis of transposition in transgenic mice, we used visible markers (Red Fluorescent Protein, RFP) instead of drug resistance markers in donor plasmids. Donor PB[Act-RFP] elements and the helper plasmid Act-PBase were co-injected in circular forms into pronuclei of FVB/Nj mouse embryos. PCR analysis showed that 34.8% (62/184) of the founders were PB[Act-RFP] single positive, 0.5% (1/184) were Act-PBase single positive, and 2.7% (5/184) were doubly positive. In comparison, only 10.4% (10/96) of the pups were positive when injection was carried out with PB[Act-RFP] alone. Similar results were obtained when a longer PB element with a different marker gene, tyrosinase, which affects skin pigmentation, PB[K14-Tyr], was co-injected with the same helper construct (FIG. 1 and FIG. 3A).

To analyze the structures of integrated transgenes in RFP positive founders, Southern hybridization with a transposon specific probe was performed (FIG. 1A). The majority of the founders carried multiple integration events (FIG. 3B). We then performed inverse PCR to recover genomic sequences flanking transposon termini. A total of 85 transposition events were recovered from 42 RFP positive founders (Table 4).

TABLE 4
PB transposition in mice.

| | | | | |
|---|---|---|---|---|
| Insertion No. | Insertion Site | Chromosome | Gene Name/Ensemble ID | Insertion Position |

| | | | | |
|---|---|---|---|---|
| AFO-82T22 | TTAAGCAAGGTCAC | 1 | | intergenic |
| | (SEQ ID NO: 34) | | | |
| AFO-168T18 | TTAAAGGCATGGAC | 1 | Gtl6 | intron |
| | (SEQ ID NO: 35) | | | |
| CFO-61T70 | TTAGTGATGCCTAc | 1 | NM_177835 | intron |
| | (SEQ ID NO: 36) | | | |
| CFO-71T61 | TTAAGGAGAAAAAG | 1 | Stau2 | intron |
| | (SEQ ID NO: 37) | | | |
| AF1-38T20 | TTAAATAAAATGTC | 2 | Trpm7 | exon (3′UTR) |
| | (SEQ ID NO: 38) | | | |
| AFO-11T2 | TTAAGCTTTTCGTT | 2 | Ssrp1 | intron |
| | (SEQ ID NO: 39) | | | |
| AFO-40T20 | TTAAAGGGACCTTG | 2 | Sh2d3c | intron |
| | (SEQ ID NO: 40) | | | |
| CFO-70T55 | TTAAACATGTTCTG | 2 | NM_177727 | intron |
| | (SEQ ID NO: 41) | | | |
| AFO-24T5 | TTAATCCCAGCACT | 2 | GENSCAN00000062119 | intron |
| | (SEQ ID NO: 42) | | | |
| AFO-38T15 | TTAACATTCCAGAC | 3 | | intergenic² |
| | (SEQ ID NO: 43) | | | |
| AFO-83T16 | TTAAAACTAGCTGT | 3 | | intergenic² |
| | (SEQ ID NO: 44) | | | |
| AFO-180T25 | TTAAAATTCTGGGA | 3 | Ashl1 | intron |
| | (SEQ ID NO: 45) | | | |
| DFO-18T60 | TTAAGTGGGAAAGT | 3 | Madh9 | intron |
| | (SEQ ID NO: 46) | | | |
| AFO-46T18 | TTAAATATATGAAG | 3 | GENSCAN00000124364 | intron |
| | (SEQ ID NO: 47) | | | |
| AFO-70T2 | TTAAAGAAATAAAC | 3 | GENSCAN00000082627 | intron |
| | (SEQ ID NO: 48) | | | |
| AFO-34T4 | TTAAAAAATAATTC | 4 | 4930523M17Rik | intron |
| | (SEQ ID NO: 49) | | | |
| CFO-70T56 | TTAAGAACACAGGT | 4 | ENSMUSESTT0000035443 | intron |
| | (SEQ ID NO: 50) | | | |
| DFO-9T22 | TTAACAAATGTTTG | 4 | BNSMUSESTT0000065097 | intron |
| | (SEQ ID NO: 51) | | | |
| AFO-153T10 | TTAAAGGAAATAAG | 4 | GENSCAN00000023389 | intron |
| | (SEQ ID NO: 52) | | | |
| AFO-50T15 | TTAACAAGAGCTGA | 4 | GEN3CAN00000064724 | intron |
| | (SEQ ID NO: 53) | | | |
| CFO-61T74 | TTAACAGAGGCAGC | 4 | | intergenic³ |
| | (SEQ ID NO: 54) | | | |
| AFQ-51T3 | TTAAGATGTGTGTG | 4 | | intergenic³ |
| | (SEQ ID NO: 55) | | | |
| AFO-180T5 | TTAAAATCCTACAA | 4 | | intergenic^{2,3} |
| | (SEQ ID NO: 56) | | | |
| AFO-90T3 | TTAAGCTTAACTGC | 5 | | intergene |
| | (SEQ ID NO: 57) | | | |
| AFO-47T6 | TTAAATTGCCTTCC | 5 | pkd2 | intron |
| | (SEQ ID NO: 58) | | | |
| AFO-40T7 | TTAAAGAACAACAT | 5 | GENSCAN00000122670 | intron |
| | (SEQ ID NO: 59) | | | |
| AFO-90T12 | TTAAGAATACATAC | 6 | | intergenic² |
| | (SEQ ID NO: 60) | | | |
| AFO-53T11 | TTAATATCTGCTAT | 6 | Osbp13 | intron |
| | (SEQ ID NO: 61) | | | |
| AFO-82T1 | TTAAGGAGGAAAGG | 6 | ENSMUSG00000029797 | intron |
| | (SEQ ID NO: 62) | | | |
| AFO-90T30 | TTAAGAGGAAATCG | 6 | Mgl1 | intron |
| | (SEQ ID NO: 63) | | | |
| AFO-41T17 | TTAAAATATCTTAG | 6 | St7 | intron |
| | (SEQ ID NO: 64) | | | |
| AFO-24T12 | TTAAATAAATTTAA | 6 | ENSMUSESTT00000055704 | intron |
| | (SEQ ID NO: 65) | | | |
| AFO-87T1 | TTAAATAGTAGAAA | 6 | ENSMUSESTT00000044094 | intron |
| | (SEQ ID NO: 66) | | | |
| AFO-38T7 | TTAAGGCTAAGAAT | 6 | | intergenic³ |
| | (SEQ ID NO: 67) | | | |
| AFO-81T10 | TTAAAAGCAGCATT | 7 | | intergenic² |
| | (SEQ ID NO: 68) | | | |
| CFO-61T68 | TTAAAAATTAATTG | 7 | | intergenic |
| | (SEQ ID NO: 69) | | | |
| AFO-81T2 | TTAAAGTCATGTAA | 7 | | intergenic^{2,3} |
| | (SEQ ID NO: 70) | | | |
| AFO-81MT43 | GTTAAAGCATTTAA | 7 | | intergenic² |
| | (SEQ ID NO: 71) | | | |
| AFO-142T5 | TTAAGGAGAAAGAT | 8 | Pmfbp1 | intron |
| | (SEQ ID NO: 72) | | | |
| AFO-70T7 | TTAAAGAACAACAA | 8 | Elav11 | intron |
| | (SEQ ID NO: 73) | | | |
| AFO-90T23 | TTAAATAGTTAAAA | 8 | 2410008G02Rik | intron |
| | (SEQ ID NO: 74) | | | |
| CFO-70T54 | TTAAATAAGAGTTG | 8 | Nfix | intron |
| | (SEQ ID NO: 75) | | | |
| AFO-70T54 | TTAATGAGTATGCA | 8 | GENSCAN0000010819 | intron |
| | (SEQ ID NO: 76) | | | |
| AFO-46T30 | TTAAACCCTTCGCC | 9 | | intergenic² |
| | (SEQ ID NO: 77) | | | |
| AFO-46T33 | TTAAGGAGGAAATA | 9 | | intergenic² |
| | (SEQ ID NO: 78) | | | |
| AFO-46T34 | TTAATGTTGAAGCA | 9 | | intergenic² |
| | (SEQ ID NO: 79) | | | |
| AFO-180T34 | TTAACCGCACTTCA | 9 | Dpp8 | intron |
| | (SE9 ID NO: 80) | | | |
| AFO-48T15 | TTAAGATTTGTAAA | 9 | GENSCAN00000135754 | intron |
| | (SEQ ID NO: 81) | | | |
| AFO-11T13 | TTAAGGGAGAAAAG | 11 | GENSCAN00000047992 | intrOn |
| | (SEQ ID NO: 82) | | | |
| AFO-140T4 | TTAAGCAGGAAGCA | 11 | ENSMUSESTT00000064726 | intron |
| | (SEQ ID NO: 83) | | | |
| BF1-30T43⁴ | TTAATAACTGTTTT | 11 | GENSCANC00000019932 | intron |
| | (SEQ ID NO: 84) | | | |
| AFO-82T24 | TTAACGAAGTCCAA | 12 | ENSMUSESTG00000013173 | intron |
| | (SEQ ID NO: 85) | | | |
| AFO-60T18 | TTAAGGCTAGACTG | 12 | GENSCAN00000070967 | intron |
| | (SEQ ID NO: 86) | | | |
| CFO-40T62 | TTAAGGAAATGACA | 12 | GENSCAN00000127032 | intron |
| | (SEQ ID NO: 87) | | | |
| AFO-62T14 | TTAAATAAAGAAC | 13 | C730024G01Rik | exon |
| | (SEQ ID NO: 88) | | | |
| AFO-50T3 | TTAATCCCAGTACT | 13 | Lga1s8 | intron |
| | (SEQ ID NO: 89) | | | |
| AFO-92T3 | TTAAAATAAACATG | 13 | Hist1h2bm | intron |
| | (SEQ ID NO: 90) | | | |
| BF1-29T6⁴ | TTAAAAATCAATTT | 13 | Auh | intron |
| | (SEQ ID NO: 91) | | | |
| AFO-52T5 | TTAAAGGTTTTTCA | 13 | 2210404D11Rik | intron |
| | (SEQ ID NO: 92) | | | |
| AFO-50T24 | TTAACCAAGATCAA | 13 | ENSMUST00000038065 | intron |
| | (SEQ ID NO: 93) | | | |
| AFO-30T10 | TTAAGATCTAAATT | 13 | GENSCAN00000024946 | intron |
| | (SEQ ID NO: 94) | | | |
| CFO-71T63 | TTAAGGTGTTTTCC | 13 | GENSCAN00000004330 | intron |
| | (SEQ ID NO: 95) | | | |
| AFO-22T9 | TTAAGATAATAATT | 13 | | intergenic |
| | (SEQ ID NO: 96) | | | |
| AFO-62T1 | TTAAATTCACGTTG | 14 | Ktn1 | intron |
| | (SEQ ID NO: 97) | | | |
| AFO-67T10 | TTAAACTTTAATCT | 14 | ENSMUSESTT00000033918 | intron |
| | (SEQ ID NO: 98) | | | |
| AFO-81T24 | TTAAGAAACTTACA | 14 | GENSCAN00000096363 | intron |
| | (SEQ ID NO: 99) | | | |
| AFO-40t15 | TTAAGGCGGAAATC | 15 | ENSMUSETT00000063224 | intron |
| | (SEQ ID NO: 100) | | | |
| AFO-136T3 | TTAAAAATATTGTT | 15 | GENSCAN00000074792 | intron |
| | (SEQ ID NO: 101) | | | |
| AFO-70T19 | TTAATAAAACATCT | 15 | GENSCAN00000038008 | intron |
| | (SEQ ID NO: 102) | | | |
| AFO-180T4 | TTAAATTTACCATA | 16 | Umps | intron |
| | (SEQ ID NO: 103) | | | |
| AFO-50T4 | TTAAATTTTCCTGG | 16 | Ufd11 | intron |
| | (SEQ ID NO: 104) | | | |
| AFO-40T4 | TTAACAACTGGGAT | 16 | GENSCAN00000132965 | intron |
| | (SEQ ID NO: 105) | | | |
| AFO-90T4 | TTAAGAGCTTTTTA | 16 | | intergenic |
| | (SEQ ID NO: 106) | | | |
| AFO-81T19 | TTAAATGAAAATTA | 17 | Birc6 | intron |
| | (SEQ ID NO: 107) | | | |
| DFO-18T69 | TTAAGAAAATGCCT | 17 | 4732490P18Rik | intron |
| | (SEQ ID NO: 108) | | | |
| AFO-86T6 | TTAAAGGTGCTCAT | 18 | Pde6a | intron |
| | (SEQ ID NO: 109) | | | |
| BF1-44T10⁴ | TTAAAAATATTAAC | 18 | Wdr7 | intron |
| | (SEQ ID NO: 110) | | | |
| AFO-90T36 | TTAAGATGGCTAAG | X | ENSMUSG00000025065 | intron |
| | (SEQ ID NO: 111) | | | |
| AFO21T14 | TTAAGTAAAAAAAA | X | ENSMSETT00000038201 | intron |
| | (SEQ ID NO: 112) | | | |
| AFO-40T21 | TTAACAGTCTATTC | X | GENSCAN00000036541 | intron |
| | (SEQ ID NO: 113) | | | |
| AFO-92T2 | TTAAGTAGTTAAGC | X | GENSCAN00000048518 | intron |
| | (SEQ ID NO: 114) | | | |
| AFO-12T5 | TTAAGGCACAATA | X | | intergenic |
| | (SEQ ID NO: 115) | | | |
| AFO-66T4 | TTAAAGAAATCATC | no hit | | |
| | (SEQ ID NO: 116) | | | |
| AFO-66T13 | TTAAACCAGGATCC | no hit | | |
| | (SEQ ID NO: 117) | | | |
| AFO-51T13 | TTAAAATACCCTTT | undefined | | |
| | (SEQ ID NO: 118) | | | |
| AFO-88T3 | TTAATGAAACCTTT | undefined | | |
| | (SEQ ID NO: 120) | | | |
| AFO-130T3 | TTAAAGAAGGAGAG | undefined | | |
| | (SEQ ID NO: 121) | | | |
| AFO-91T29 | TTAATCTTATGTCA | undefined | | |
| | (SEQ ID NO: 122) | | | |
| AFO-92T15 | TTAAGACCTTTCAT | undefined | | |
| | (SEQ ID NO: 123) | | | |
| DFO-20T24 | TTAACATACTAGAT | repeats (Alu) | | |
| | (SEQ ID NO: 124) | | | |
| AFO-40T19 | TTAAAAAAATAGAT | repeats | | |
| | (SEQ ID NO: 125) | | | |
| AFO-51T10 | TTAAAAAAAGGACA | repeats | | |
| | (SEQ ID NO: 126) | | | |
| AFO-69T4 | TTAAGGAGCATTCT | repeats | | |
| | (SEQ ID NO: 127) | (IAPLTR1-MM) | | |

^{1.}A, B: PB[Act-RFP]; C: PB[K14-Tyr, Act-RFP]; D: PB[Act-RFP, MCK-TSC I];
^{2.}Less than 10 kb downstream of known or predicted genes;
^{3.}Less than 10 kb upstream of known or predicted genes;
^{4.}The insertions from germline transpositions.

Most of these transpositions were mapped to the mouse genome according to genomic sequence flanking the right terminal repeat of the integrated transposon. We randomly selected nine transposition events and amplified the genomic junction sequences on the opposite side of the transposon. In each case, transposon insertion was found to produce a precise TTAA duplication of the integration site (data not shown). These results indicate that most of the transgene integrations produced from co-injection were due to transposition.

To test the capability of integrated transposons to transmit through the germline, several PB[Act-RFP] positive but helper plasmid negative founders were mated with wild type FVB/Nj mice to generate transgenic lines. One of the founders (AF0-61) that had eight PB[Act-RFP] integrations was analyzed in detail. PCR-based genotyping showed that 15 out of 16 progenies of this founder retained the transposon DNA. Southern analysis of PCR positive individuals showed that all of them inherited at least one copy of the transposed PB[Act-RFP] (FIG. 3C and data not shown). The random segregation of these transgenes suggested a diverse chromosomal distribution of the initial transposition events in the founder. Progeny analysis of a second founder (AF0-47) that carried a single transposon indicated that two out of eight F is in a single litter inherited the transposon (FIG. 3C and data not shown). PCR-based genotyping with primers targeting several individual transposon integration sites also confirmed stable inheritance of the integrated transposons from founders to the F1 generation (date not shown). Taken together, the high frequency of transposition-mediated gene integration and the capability of integrated transgenes to transmit through the germline demonstrates the feasibility of using piggyBac elements as gene transfer tools in the mouse.

6.3.3. Precise Excision and Transposition of PiggyBac in Mouse Germline

We further tested the transposition behavior of piggyBac in mouse germline with the classical breeding strategy of “jumpstarter” and “mutator” stocks (Cooley et al., 1988, Science 239:1121-1128; Horn et al., 2003, Genetics 163:647-661). In this procedure, a mutator line carrying a nonautonomous transposon is crossed with a jumpstarter line that expresses transposase in the male germline. Active transposition is expected to occur exclusively in the germ cells of males carrying both transposon and transposase DNA. These males are subsequently mated with wild type females to produce lines with new transposon insertions. We revised this procedure and used co-injection method to directly produce mice doubly positive for a nonautonomous transposon and a helper transposase gene. Transgenic animals were produced by conventional pronuclei injection of linear plasmids, which assured the co-integration of both donor and helper plasmids in the same locus. Several transgenic mouse lines carrying both PB[Act-RFP] and protamine 1 (prm1) promoter-driven piggyBac transposase transgenes (Prm1-PBase) were generated. The prm1 promoter was expected to be active during spermiogenesis (O'Gorman et al, 1997, Proc. Nat'l. Acad. Sci. USA 94:14602-14607). Thus, in such doubly positive transgenic lines, male mice were expected to produce new transposition events whereas female mice could be used as breeders.

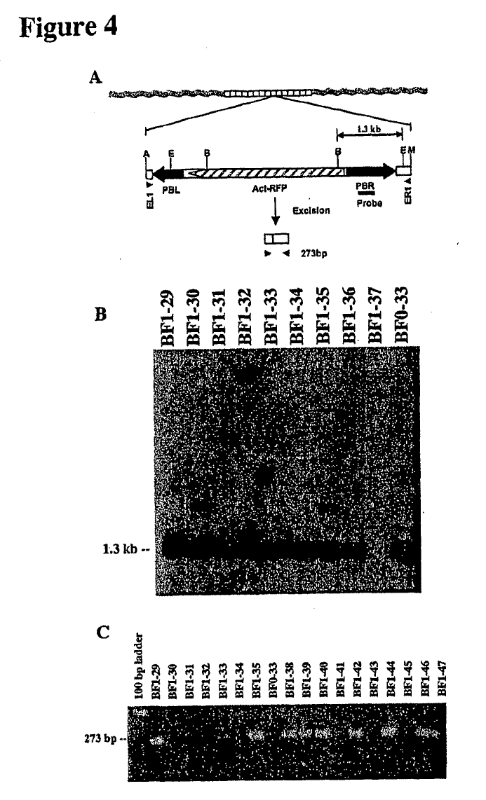

One of these double transgenic lines, referred as BF0-33, was tested for transposition in its progeny. Southern hybridization with the transposon specific primer (FIG. 1A) revealed new transposon integrations in 67.8% (19/28) of the transposon positive progenies (FIG. 4A and data not shown). On average, 1.1 new insertions were generated per gamete. The new insertions seemed not to be regional since three of those new insertions were sequenced and found to be located on three separate chromosomes (BF1-29T6, BF1-30T43, and BF1-44T10 in Table 4).

Primers targeting PB[Act-RFP] plasmid sequences flanking the transposon were used to explore the transposition behavior of piggyBac in the germline (FIG. 4B). If piggyBac transposed through a cut-and-paste manner, a 273 by PCR product would be detected. Indeed, this PCR product was detected in 10 out of 17 offspring from line BF0-33 (FIG. 4C). Seven of these samples have been sequenced and revealed the existence of a single TTAA target site (data not shown), demonstrating that piggyBac transposed through a precise cut-and-paste mechanism in male germline of mouse. Because the founder carried a transgene array, it is expected that some transposition events (progeny BF1-30 and BF1-32 in FIG. 4B-C) were not coupled with the detection of this 273 by product.

6.3.4. PiggyBac Transposon System as a Unique Transgenic Tool

It has been shown previously that transposition efficiency significantly decreases with increasing the length of some transposons, which hampers their utility as a genetic tool. For example, in Hela cells, SB transposons were shown to have an approximately 30% decrease in efficiency of transposition with each kb increase in length in addition to its 2.2 kb original length (Izsvak et al., 2000, J, Mol. Biol. 302:93-102). To determine the size limitation of PB transposition in mice, several PB elements ranging from 4.8 to 14.3 kb were used in making transgenic mice (FIG. 1A). These transposons carried either a RFP reporter cassette and/or a separate transcription unit. The integration rate of these PB elements in circular plasmids was tested in the absence or presence of Act-PBase helper plasmid (FIG. 3A). Results indicated that PB elements can carry 9.1 kb of foreign sequence without significantly reducing integration efficiency. PCR analysis confirmed the presence of transposition events in 83.9% (26/31) of the founders with the PB[K14-Tyr, Act-RFP] element, which carries two marker genes. Helper-assisted integration dropped using the 14.3 kb PB[Act-RFP, MCK-TSC1] element. Eleven PB[Act-RFP, MCK-TSC1] positive founders were analyzed by Southern hybridization and inverse PCR, and four were found to carry transposition integration (Table 4 and data not shown). Thus, PB is able to transpose sequence up to 14 kb.

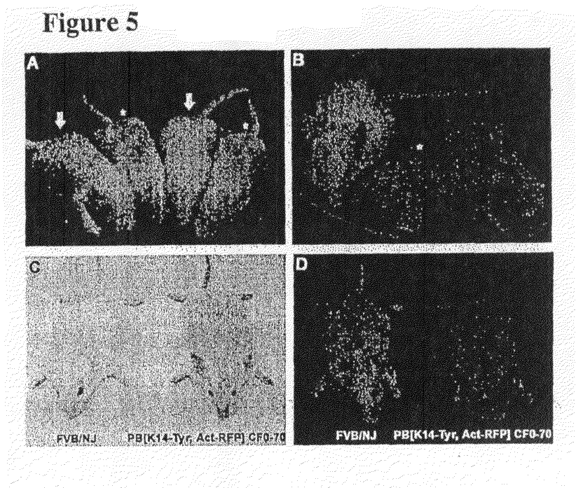

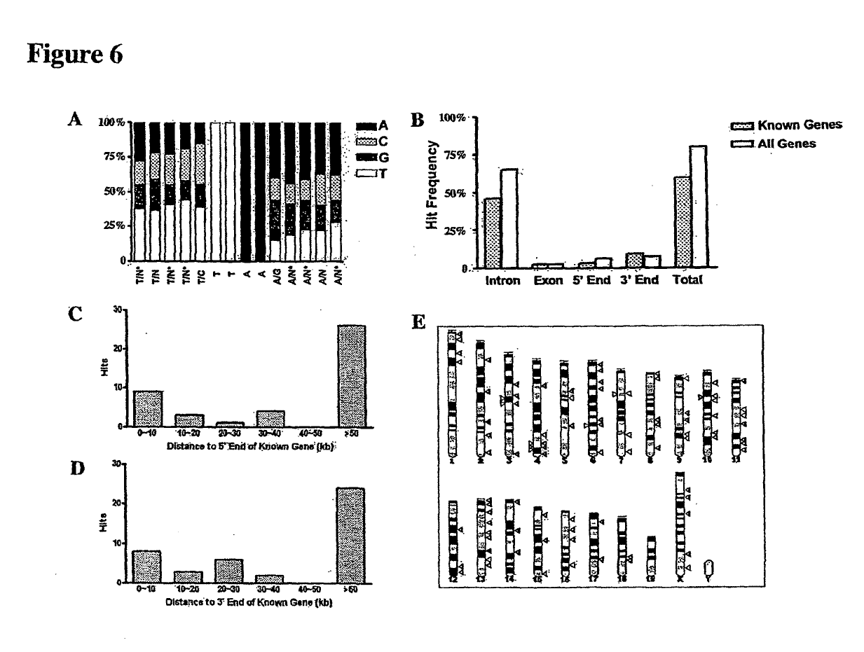

Next, we evaluated the behavior of transgene expression from integrated PB elements. Among the mice that carried PB[Act-RFP], 98% (39/40) expressed the RFP marker. In our experiment, even one copy of PB[Act-RFP] transposon produced a visible red signal under UV illumination (FIG. 5A). Some of these founders exhibit mosaic RFP signals, a phenomenon most likely due to the transposition in embryonic development after the one-cell stage (FIG. 5B). Co-expression of both RFP and tyrosinase markers was observed in twenty-nine percent (9/31) of the founders carrying PB[K14-Tyr, Act-RFP], a transposon containing both a K14 promoter-driven tyrosinase gene (K14-tyr) and a RFP expression cassette (FIGS. 1A, 5C and 5D). Thus, the PB[Act-RFP] construct, which contains unique cloning sites and a RFP marker, serves as a universal transgenic PB vector. The ability of simultaneous expression of two separate transcription units and high frequency integration events suggests that PB transposition can be used as an effective method to generate transgenic mice.

6.3.5. PiggyBac Transposon System as an Insertional Mutagenesis Tool

To test the feasibility of PB as an insertional mutagenesis tool in vertebrates, we evaluated 104 transposition events produced in mice (Table 3). First, the TTAA sequence was found at all PB integration sites except one. Second, we compared the genomic sequences flanking the TTAA sites of integration with randomly sampled TTAA sites in the mouse genome and found enrichment of Ts and As surrounding the core TTAA sequence (FIG. 6A). This is similar to the integration sites found in insects (Li et al., 2005, Insect Mol Biol. 14(1):17-30.). Finally, genomic locations of these transposition sites were analyzed against the Ensembl mouse genome database. Although some of the sites could not be mapped due to the presence of repetitive sequences and sequence gaps in the database, the exact locations of 93 transposon integration sites were determined (Table 4, FIG. 6E). A wide range of chromosomal distribution was observed among these transposition sites. All mouse chromosomes except two (chromosome 19 and chromosome Y) were hit by PB transpositions (FIG. 6E).

Sixty-seven percent (70/104) of all transposition sites were mapped to known or predicted transcription units. Among these integrations, about 97% (68/70) hit introns, while 3% (2/70) hit exons (FIG. 6B). The preference of integration within transcription units still remained high even if unvalidated (i.e., predicted) genes and ESTs were excluded from analysis (48% (50/104)). Furthermore, more than 40% of the “intergenic” transpositions were mapped within 50 Kb of known genes or ESTs (FIGS. 6C and 6D). When a 10 Kb interval was set as an arbitrary threshold for regulatory regions at 5′ and 3′ ends of a transcription unit, the frequency of genes hit by PB transposition were about 80% (83/104) for known or predicted transcription units (FIG. 6B). The wide chromosomal distribution and the preference of transposition into transcription units indicates that PB elements can be used as a highly effective mutagen for genome-wide genetic screens.

Additional studies, for up to a total of 128 new insertions, show that 112 are, located in transcription units, covering all chromosomes. 5 of the transposons map to exons, and 63 to introns.

### 6.4. Discussion

We have shown that PB elements can actively transpose in mouse and human cells. PB transposition has been thought to be less dependent on host factors than other transposons, for it is the only known transposon capable of transposition in more than a dozen different insect species (Handler, 2002, Insect Biochemistry & Molecular Biology 32:1211-1220; Sumitani et al., 2003, Insect Biochem. Mol. Biol. 33:449-458). The fact that PB can effectively transpose in both insects and mammals indicates that this transposon system can have broad applications for genetic studies in both invertebrates and vertebrates. It further suggests that the transposition mechanism of PB elements may be significantly different from other naturally existing transposons, which only work in highly restricted species.

6.4.1. PiggyBac as a Tool for Transgenesis

Our studies suggest that PB is a practical tool for generating transgenic mice and perhaps for generating other transgenic vertebrate animals. First, PB can be introduced into the mouse germline with high efficiency. Pronuclear co-injecting of helper and donor plasmids results in more than 30% of the donors carrying integrated donor plasmids in their germline (FIG. 3A). Second, the approach produces single copies of integrated transgenes. In most cases, classical pronuclear injection of linear DNA into mice results in the formation of transgene concatamers (Nagy et al., 2003, Manipulating the mouse embryo: a laboratory manual, 3rd edition (Cold Spring Harbor Laboratory Press)). We showed that individual transposon integration sites can be quickly defined by inverse PCR. Thus the effect of the chromatin environment on integrated transgenes can be estimated. Third, the PB element allows the expression of the transgene it carries. The overall frequency of mice showing the expected transgenic expression pattern was comparable to conventional transgenic experiments. Finally, our results indicate that PB can carry transgenes up to 9.1 kb without a significant reduction of the transposition frequency. Transposition was observed for transgenes as big as 14.3 kb, which allows insertions much bigger than retroviral vectors can carry. Thus, a single PB element can carry multiple genes, which allows one to perform complex transgenic experiments such as identifying positive transgenic animals with the help of a visible marker.

6.4.2. PiggyBac as a Genomics Tool Deciphering Gene Function

In the post-genome era, systematic gene inactivation is one of the most powerful approaches to decipher the function of the genome. This approach has been proven to be successful in the study of single cell organisms like bacteria and yeast, as well as of multi-cellular organisms such as C. elegans, Drosophila, zebrafish, and Arabidopsis. Unfortunately, efficient methods for genome-wide gene inactivation in mammals are still limited. ENU mutagenesis is one of the few available methods for genome-scale gene inactivation in the mouse; however, mapping ENU-induced mutations and cloning the genes defined by the mutations is usually laborious and time consuming (Herron et al., 2002, Nat. Genet. 30:185-189). Retroviral-mediated insertional mutagenesis has also been widely used to produce mutations throughout the mouse genome. While this method indeed produces a large number of mutations, most of these mutations are generated in mouse ES cells, and a significant amount of additional effort is needed to transmit these gene specific mutations into live animals. Recently, SB has been tested for the insertional mutagenesis in the mouse. However, local hopping and a relatively low efficiency of transposition into transcription units prevent it from being widely used.

In contrast, PB provides a new and attractive choice for screening recessive mutations in the mouse. The success of efficient PB transposition in the mouse germline suggests the suitability of this transposon for insertional mutagenesis. Several unique properties of PB could greatly facilitate insertional mutagenesis studies in mice. One important consideration of insertional mutagenesis experiments is whether the mutagen can hit every gene in the genome in an unbiased fashion. Our experiments have shown that PB integrations have a diverse distribution in the mouse genome, which is consistent with a recent study in Drosophila showing that PB hits genes in a less biased fashion than the widely used P-element (Thibault et al., 2004, Nat. Genet. 36:283-287).

Interestingly, our study has revealed a high preference of PB transposition for transcriptional units. 67% of the transposon integrations were found within known or predicted transcriptional units. Including insertions in the regulatory regions adjacent to the transcriptional initiation and termination sites, the frequency of PB transposition in genes is even higher. Given that only ˜15% of the mouse euchromatin sequence encodes genes, PB transposition is highly selective for coding sequences. It is not clear whether this integration property is influenced by the transcriptional activities of the genome or the exogenous sequence carried by the PB elements. Nevertheless, this integration preference makes PB a potential dream tool for genome-wide insertional mutagenesis.

An important aspect in the analysis of mutations obtained from random mutagenesis is the verification of the relationship between mutations and the phenotypes they cause. This is particularly important in the analysis of novel genes. Verification of genotype/phenotype correlation is usually done by introducing a wild-type gene into the mutant background and looking for phenotypic “rescue” (ideally, reversion of the induced mutation back to wild-type). Another way to determine genotype/phenotype correlation is to excise insertional mutations and look for phenotypic reversion. The ability of transposons to excise has thus always been considered as an important advantage over retriviral vectors. However, most transposons leave a small deletion or insertion after excision from the original site. Interestingly, PB generally leaves no footprint after excision, making it ideal for producing revertants. This feature also makes PB less likely to cause genomic damage during mutagenesis, in which multiple transposition events occur in a single genome. Our studies have demonstrated that PB excision can be easily achieved with germline expression of the transposase. The fact that PB can carry multiple genes during transposition offers great advantages for many genetic manipulations including insertional mutagenesis and phenotypic characterization. It allows one to follow the insertion/mutation and the status of the mutation, such as heterozygous versus homozygous and single mutant versus double mutants, by visible markers such as RFP and Tyrosinase. Given the long generation time and the high animal housing cost associated with mouse breeding, this will dramatically cut down the cost for many types of experiments and will make some unrealistic experiments become practical.

Furthermore, PB transposons for insertional mutagenesis could also carry reporter genes for enhancer/promoter detection, or “gene trapping”, which can greatly facilitate the effort of functional annotation of the mouse genome and provide reagents for many types of biological analyses. For example, the gene trap technology can be used with the PB system. Microinjection or crossing can be used to induce the PB transposons carrying the gene trap vector transpose into the mouse genome. When the transposon inserts into the introns of the genes in the right direction, the marker gene (eg. LacZ) in it will be activated and the endogenous gene will be disrupted. This allows both the detection of the reporter expression and, in some instances, visible phenotypes caused by the gene disruptions in some of the trapped lines.

In conclusion, our experiments provide a the basis for a highly efficient transgenesis and insertional mutagenesis system in mouse, and suggest that the PB system can also be used as a powerful tool for genetic manipulations in other vertebrate organisms. (Thibault et al., 2004, Nat. Genet. 36:283-287).

### 7. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A method of generating a transgenic non-human vertebrate comprising in the genome of one or more of its cells a piggyBac-like transposon which carries an insert of at least 1.5 kb, comprising the steps of:
(a) introducing ex vivo into a non-human vertebrate embryo or fertilized oocyte a nucleic acid comprising a piggyBac-like transposon which carries an insert of at least 1.5 kb and, within the same or on a separate nucleic acid, a nucleotide sequence encoding a piggyBac-like transposase;
(b) implanting the resultant non-human vertebrate embryo or fertilized oocyte into a foster mother of the same species under conditions favoring development of said embryo into a transgenic non-human vertebrate; and
(c) after a period of time sufficient to allow development of said embryo into a transgenic non-human vertebrate, recovering the transgenic non-human vertebrate from the mother;
thereby generating a transgenic non-human vertebrate comprising in the genome of one or more of its cells piggyBac-like transposon which carries an insert of at least 1.5 kb.

2. The method of claim 1, wherein the piggyBac-like transposon comprises a nucleotide sequence encoding a protein that modifies a trait in said transgenic non-human vertebrate.

3. The method of claim 1, wherein said nucleic acid comprising the piggyBac-like transposon is linearized, such that the genome of one or more of said cells comprises said piggyBac-like transposon within a concatamer, said concatamer comprising a plurality of piggyBac-like transposons.

4. The method of claim 2, wherein said nucleic acid comprising the piggyBac-like transposon is linearized, such that the genome of one or more of said cells comprises said piggyBac-like transposon within a concatamer, said concatamer comprising a plurality of piggyBac-like transposons.

5. The method of any one of claims 1-4, wherein the piggyBac-like transposon comprises a sequence recognized by a protein that binds to and/or modifies nucleic acids.

6. The method of claim 5, wherein the nucleic acid-modifying protein is a DNA-binding protein, a DNA-modifying protein, an RNA-binding protein, or an RNA-modifying protein.

7. The method of any one of claims 5, wherein the piggyBac-like transposon comprises a target site for a site-specific recombinase.

8. The method of claim 7, wherein the target site is a FRT target site or a lox target site.

9. The method of any one of claims 1-4, wherein the piggyBac-like transposon comprises a selectable marker.

10. The method of any one of claims 1-4, wherein the piggyBac-like transposon comprises a reporter gene.

11. The method of claim 10, wherein the reporter gene is endogenous to the species of said species.

12. The method of any one of claims 1-4, wherein the piggyBac-like transposon comprises both a selectable marker and a reporter gene.

13. The method of any one of claims 1-4, wherein the piggyBac-like transposon and the nucleotide sequence encoding the piggyBac-like transposase are within the same nucleic acid.

14. The method of any one of claims 1-4, wherein the piggyBac-like transposon and the nucleotide sequence encoding the piggyBac-like transposase are on separate nucleic acids.

15. The method of claim 14, wherein the nucleic acid comprising the piggyBac-like transposon is DNA and the nucleic acid comprising the piggyBac-like transposase is RNA.

16. The method of claim 15, wherein the piggyBac-like transposon is immobilized in said non-human vertebrate.

17. The method of claim 14, wherein the nucleic acids comprising the piggyBac-like transposon and the piggyBac-like transposase are both DNA.

18. The method of claim 17, wherein the transgenic non-human vertebrate further comprises in the genome of one or more of its cells nucleotide sequence encoding a piggyBac-like transposase.

19. The method of claim 18, wherein the nucleotide sequence encoding the piggyBac-like transposase is operably linked to a promoter.

20. The method of claim 19, wherein the promoter directs expression of the transposase in the germline.

21. The method of claim 20, wherein the promoter is a germline-specific promoter.

22. The method of claim 18, wherein the genome of one or more of said cells comprises said nucleotide sequence encoding the piggyBac-like transposase within a concatamer, said concatamer comprising a plurality of nucleotide sequence, each of which encodes a piggyBac-like transposase.

23. The method of claim 1, wherein the non-human vertebrate is a non-human mammal.

24. The method of claim 1, wherein the non-human vertebrate is a livestock animal.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. A method of generating a recombinant vertebrate cell in culture whose genome comprises a piggyBac-like transposon which carries an insert of at least 1.5 kb, comprising the steps of:
(a) introducing into a vertebrate cell in culture a nucleic acid comprising a piggyBac-like transposon which carries an insert of at least 1.5 kb, and, within the same or on a separate nucleic acid, a nucleotide sequence encoding a piggyBac-like transposase: and
(b) culturing said cell under conditions in which the piggyBac-like transposase is expressed such the piggyBac-like transposon is integrated into the genome of said vertebrate cell in culture,
thereby generating a recombinant vertebrate cell in culture whose genome comprises a piggyBac-like transposon which carries an insert of at least 1.5 kb.

81. The method of claim 80 wherein the piggyBac-like transposon comprises a nucleotide sequence encoding a protein of value in the treatment or prevention of a vertebrate disease or disorder
(a)
(b).

82. (canceled)

83. (canceled)

84. The method of any one of claims 80 and 81, wherein the vertebrate cell is a mammalian cell.

85. The method of claim 84, wherein the mammalian cell is a human cell.

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. A transgenic non-human vertebrate comprising in the genome of one or more of its cells a piggyBac-like transposon which carries an insert of at least 1.5 kb.

107. The transgenic non-human vertebrate of claim 106 wherein the piggyBac-like transposon comprises a nucleotide sequence encoding a protein that modifies a trait in said transgenic non-human vertebrate.

108. (canceled)

109. A vertebrate cell in culture comprising in its genome a piggyBac-like transposon which carries an insert of at least 1.5 kb.

110. The vertebrate cell of claim 109 wherein the piggyBac-like transposon comprises a nucleotide sequence encoding a protein of value in the treatment or prevention of the vertebrate disease or disorder.

111. The vertebrate cell of claim 109 wherein the piggyBac-like transposon comprises a nucleotide sequence encoding a protein that modifies a trait in a transgenic non-human vertebrate.

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. The method of claim 1, wherein step (a) comprises introducing into said non-human vertebrate embryo or fertilized oocyte a recombinant virus whose genome comprises (i) the piggyBac-like transposon and (ii) the nucleotide sequence encoding a piggyBac-like transposase operably linked to a promoter.

129. The method of claim 128, wherein the virus is a retrovirus, an adenovirus, or an adeno-associated virus.

130. (canceled)

131. (canceled)

132. (canceled)

133. (canceled)

134. (canceled)

135. (canceled)

136. (canceled)

137. The method of any one of claim 1, 80, 81, or 128, wherein the piggyBac-like transposon is a piggyBac transposon, and/or the piggyBac-like transposase is a piggyBac transposase.

138. The transgenic non-human vertebrate of any one of claims 106-107, wherein the piggyBac-like transposon is a piggyBac transposon, and/or the piggyBac-like transposase is a piggyBac transposase.

139. (canceled)

140. The vertebrate cell of any one of claims 109-111, wherein the piggyBac-like transposon is a piggyBac transposon, and/or the piggyBac-like transposase is a piggyBac transposase.

141. (canceled)

142. (canceled)

143. The method of any one of claims 1 and 80 wherein the piggyBac-like transposon carries an insert of at least 4 kb.

144. The transgenic non-human vertebrate of claim 106, wherein the piggyBac-like transposon carries an insert of at least 4 kb.

145. (canceled)

146. The vertebrate cell of claim 109, wherein the piggyBac-like transposon carries an insert of at least 4 kb.

147. (canceled)

148. (canceled)

149. The method of claim 21, wherein the germline specific promoter is a male-specific promoter.

150. The method of claim 149, wherein the male-specific promoter is a Protamine (Prm) promoter.

151. The method of claim 21, wherein the germline specific promoter is a female-specific promoter.

152. The method of claim 151, wherein the female-specific promoter is a ZP3 promoter.

153. A nucleic acid comprising a piggyBac-like transposon which carries an insert of at least 1.5 kb, wherein the insert comprises a vertebrate promoter.

154. A nucleic acid comprising a nucleotide sequence encoding a piggyBac-like transposase operatively linked to a vertebrate promoter.
